(19) United States
(12) Patent Application Publication
(10) Pub. No.: US 2026/0234282 A1
(43) Pub. Date: Aug. 13, 2026

(54) CD163-BINDING CONJUGATES

(71) Applicants: VIB VZW, Gent (BE); Vrije Universiteit Brussel, Brussel (BE)

(72) Inventors: Jo Van Ginderachter, Brussel (BE); Geert Raes, Brussel (BE); Cécile Vincke, Brussel (BE); Nick Devoogdt, Brussel (BE); Timo De Groof, Brussel (BE); Yoline Lauwers, Brussel (BE)

(21) Appl. No.: 19/148,156

(22) PCT Filed: Jan. 26, 2024
(86) PCT No.: PCT/EP2024/051945

§ 371 Date: Jul. 15, 2025

(30) Foreign Application Priority Data
Jan. 27, 2023 (EP) 23153695.4
Jan. 27, 2023 (EP) 23153700.2
Aug. 28, 2023 (EP) 23193741.8
Aug. 28, 2023 (EP) 23193745.9

(51) Int. Cl. C07K 16/28; A61K 47/68; A61K 51/08

(52) U.S. Cl. CPC C07K 16/2896; A61K 47/6849; A61K 51/088; A61K 2123/00; C07K 2317/24; C07K 2317/565; C07K 2317/567; C07K 2317/569; C07K 2317/92; C07K 2319/21

(57) ABSTRACT

The invention relates to polypeptides, in particular polypeptides comprising an immunoglobulin domain, binding to human and murine CD163 protein and to applications of such polypeptides such as for use as diagnostic agent, for example as an immunotracer. The invention further relates to conjugates, in particular conjugates of polypeptides comprising an immunoglobulin domain binding to human and murine CD163 protein and a further moiety, such as a therapeutic moiety, and to applications of such conjugates.

### FIELD OF THE INVENTION

The invention relates to polypeptides, in particular polypeptides comprising an immunoglobulin domain, binding to human and murine CD163 protein and to applications of such polypeptides such as for use as diagnostic agent, for example as an immunotracer. The invention further relates to conjugates, in particular conjugates of polypeptides comprising an immunoglobulin domain binding to human and murine CD163 protein and a further moiety, such as a therapeutic moiety, and to applications of such conjugates.

### BACKGROUND

Immunomodulating agents, and immune checkpoint inhibitors (ICIs) in particular, have revolutionized cancer treatment. Although highly effective in a subset of cancers and cancer patients, ICIs remain ineffective in a relative large fraction of cancers and cancer patients. Many efforts are now devoted to understanding the underlying inferior response or non-response to ICI therapy, and to finding ways to break poor response to ICI therapy. Prediction or follow-up of response of a cancer patient to ICI therapy, or to therapy including an immunomodulating agent in general, prior to or during the therapy would be ideal both for the patient (in terms of helping in finding the best possible therapy) and in socio-economic terms (helping in optimal allocation of available healthcare funding). Therapy prediction or follow up, using invasive methods such as biopsies or blood biomarkers, has, however, so far proven to be difficult as they do not provide a complete overview of the tumor micro-environment and/or lack spatial information. Therefore, a valuable alternative would be to have non-invasive means for detecting response or non-response to therapy, i.e. a means for detecting therapeutic outcome, as early as possible after start of the therapy.

Non-invasive immune-monitoring imaging applicable for in vivo monitoring of immune responses provides a means for helping in detecting therapeutic outcome and in understanding reasons for response or non-response to a therapy of interest. Many different immune-monitoring imaging techniques are being pursued, one of which being antibody-based tracers as part of the group of molecular tracers (reviewed in e.g. McCarthy et al. 2020, Front Immunol 11:1067).

Cluster of differentiation protein 163 (CD163) is a protein of the scavenger receptor cysteine-rich (SRCR) superfamily, and known to be expressed in monocytes and macrophages. CD163 is an endocytic receptor for haemoglobin-haptoglobin-complexes. Another CD protein, CD206 (or macrophage mannose receptor, MMR) is also expressed in macrophages. The presence of tumor-associated macrophages (TAMs) in tumors is usually correlated with poor patient outcome. TAMs can represent up to 50% of a tumor mass.

Efforts in using CD206 antibody-based tracers have been reported, e.g. as early marker of tumor relapse and metastasis (Zhang et al. 2017, Theranostics 7:4276-4288), or e.g. to track reparatory macrophages in the myocardium after myocardial infarction (Varasteh et al. 2022, Front Cardiovasc Med 9:889963). A clinical trial using a CD206 single domain antibody-based tracer has been reported for positron emission tomographic imaging (PET/CT scan) of breast cancer, head and neck cancer or melanoma (NCT04168528). Much of the work on PET-tracers developed for in vivo imaging of macrophages in different disease contexts is summarized by Fernandes et al. 2022 (EJNMMI Radiopharmacy and Chemistry 7:11), with one study involving a CD163 antibody-based tracer as means to track macrophages in a collagen-induced arthritis model as example of an inflammatory disease (Eichendorff et al. 2015, Mol Imaging Biol 17:87-93). Tarin et al. 2015 (Scientific Reports 5:17135) disclosed gold-coated iron oxide nanoparticles guided to macrophages by an anti-CD163 antibody for the purpose of detecting atherosclerosis by means of MRI. Clearly, there is a need to expand the armamentarium for non-invasive immune-monitoring, especially in the cancer field.

### SUMMARY OF THE INVENTION

The invention relates to polypeptides binding to human and murine CD163, wherein the amino acid sequence of the polypeptides is comprising a CDR1 region, a CDR2 region, and a CDR3 region, wherein the CDR1, CDR2 and CDR3 regions are selected from those CDR1, CDR2 and CDR3 regions as present in an immunoglobulin variable domain (IVD) defined by SEQ ID NO:1 and as determined by the Kabat, Chothia, Martin, or IMTG method. In one embodiment, the CDR1 region is defined by SEQ ID NO:2, the CDR2 region is defined by SEQ ID NO:3, and the CDR3 region is defined by SEQ ID NO:4. In another embodiment, polypeptides binding to human and murine CD163 are further comprising at least an FR1, FR2, FR3, or FR4 region as present in an IVD, more particular as present in the IVD defined by SEQ ID NO: 1; in particular, such FR1 region is defined by SEQ ID NO:5, such FR2 region is defined by SEQ ID NO:6, such FR3 region is defined by SEQ ID NO:7, and such FR4 region is defined by SEQ ID NO:8. In a further embodiment, the above-mentioned CDR and/or FR regions are humanized and/or the above-mentioned IVD is humanized.

In a further embodiment, any of the above-defined polypeptides can be further comprising a functional moiety; in particular such functional moiety can be a His-tag or a peptide motif recognized by a peptide ligase, or can be a detectable moiety. In a particular embodiment thereto, such detectable moiety can be linked to a specific site comprised in the polypeptide.

The invention further relates to isolated nucleic acids encoding a polypeptide as defined above; to vectors comprising such nucleic acid; and to host cells expressing a polypeptide as defined above, or comprising a nucleic acid encoding a polypeptide as defined above, or comprising a vector comprising such nucleic acid.

The invention further also relates to pharmaceutical compositions comprising a polypeptide as defined above.

The invention further relates to polypeptides as defined above, or to pharmaceutical composition comprising such polypeptides, for use in diagnosis, for use in surgery, for use in therapy monitoring, or for use as an imaging agent.

The invention further relates to methods for producing a polypeptide as defined above comprising:
expressing the polypeptide in a host cell as defined above, or synthetic manufacture of the polypeptide;
purifying the expressed or manufactured polypeptide; and
optionally, coupling a detectable moiety to the purified polypeptide.

The invention further relates to polypeptides binding to human and murine CD163 which are conjugated to a prophylactic or therapeutic drug, to a cytotoxic moiety or drug, to an immunostimulatory or immunosuppressive agent, to a Toll-like receptor agonist, to a photon absorber, to a liposome or to a nanoparticle. In one embodiment, such ISVD-conjugates are for use as medicament, such as for use in treating or inhibiting cancer (choosing the appropriate payload) optionally, in combination with a further anti-cancer agent. Pharmaceutical compositions comprising such conjugates are likewise covered.

### DESCRIPTION TO THE FIGURES

FIG. 1. Affinity of the CD163-targeting single domain antibody (23766) to hCD163 and mCD163. A-B) Surface plasmon resonance plots of the binding of different concentrations of the single domain antibody 23766.

FIG. 2. Binding of the CD163-targeting single domain antibody (23766) to HEK293T cells overexpressing the hCD163 or mCD163 protein. A) Binding of different concentrations of the CD163-specific single domain antibody 23766 or an irrelevant single domain antibody (“Irr Nb”; R3b23) to HEK293T cells overexpressing (A), hCD163 or (B) mCD163 protein. Binding was detected as mean fluorescent intensity (MFI) via the C-terminal His-tag and a fluorescent labeled anti-His antibody using flow cytometry.

FIG. 3. SPECT/CT imaging of ^{99m}Tc-labeled single domain antibody 23766. Representative SPECT/CT images of C57BL/6J WT or CD163^{-/-} mice intravenously injected with ^{99m}Tc-labeled single domain antibody 23766 (“Nb 23766”) or an irrelevant single domain antibody (“Irrelevant Nb”). A high accumulation of the single domain antibody 23766 is seen in cervical lymph nodes (upper arrow), liver (middle arrow) and bone marrow (lower arrow) in WT mice.

FIG. 4. Ex vivo biodistribution analysis of the cross-reactive CD163-targeting single domain antibody 23766 in naïve animals. Ex vivo γ-counting of the isolated organs from naïve C57BL/6 WT mice and C57BL/6J CD163 KO mice 90 min after injection with ^{99m}Tc-labeled lead CD163-targeting single domain antibody or irrelevant single domain antibody (“Irr Nb”, R3b23). Biodistribution of the CD163-specific single domain antibody in three mice is shown and expressed as mean±SD of percentage of injected activity per gram of organ or tissue (% IA/g). Statistical analysis was conducted using a Student's t-test. ns, p>0.05; **, p<0.01; ***, p<0.001; ****, p<0.0001.

FIG. 5. SPECT/CT imaging of ^{99m}Tc-labeled single domain antibody 23766 in macrophage depleted and untreated naïve WT mice. Representative SPECT/CT images of treated (600 mg/kg PLX3397 in food) WT or untreated WT mice intravenously injected with ^{99m}Tc-labeled single domain antibody 23766 (“Nb 23766”), anti-MMR single domain antibody (“anti-MMR Nb”) and irrelevant single domain antibody (“Irrelevant Nb”, R3b23). In both untreated and macrophage depleted mice injected with the irrelevant single domain antibody, no accumulation in the organs except for the kidneys and bladder was observed. Both the single domain antibody 23766 and the anti-MMR single domain antibody show high accumulation in cervical lymph nodes (upper arrow), liver (middle arrow) and bone marrow (lower arrow) in WT mice that were not depleted. No or very little signal uptake is seen in the lymph nodes and liver of mice that were depleted and injected with the ^{99m}Tc-labeled single domain antibody 23766, meaning that this single domain antibody is strictly macrophage dependent. High liver uptake is seen in macrophage depleted WT mice injected with the ^{99m}Tc-labeled anti-MMR single domain antibody showing that the MMR receptor does not only express on macrophages but also on other cell types in the liver, such as liver endothelial cells.

FIG. 6. SPECT/CT imaging of the ^{99m}Tc-labeled single domain antibody 23766 in MC38-tumor, B16-F10 tumor or LLC-OVA tumor bearing mice. Representative whole-body SPECT/CT images of A) MC38-tumor bearing mice, B) B16-F10-tumor bearing mice and C) LLC-OVA-tumor bearing mice intravenously injected with ^{99m}Tc-labeled single domain antibody 23766 (“Nb 23766”) or irrelevant single domain antibody (“Irrelevant Nb”). Uptake of both single domain antibody 23766 is seen in lymph nodes (upper arrow), liver (middle arrow) and bone marrow (lower arrow) in MC38, B16-F10 and LLC-OVA tumor bearing mice. No or very little accumulation of the CD163-specific single domain antibody is shown in the MC38 tumor, only uptake in the periphery of the single domain antibody is seen in the B16-F10 tumors, and tumor uptake in the center of the tumor is seen for the single domain antibody in the LLC-OVA tumor model. D) Transverse sections of the LLC-OVA tumor intravenously injected with ^{99m}Tc-labeled single domain antibody 23766 or irrelevant single domain antibody. Tumors are delineated by a dotted line.

FIG. 7. Ex vivo biodistribution analysis of the CD163-specific single domain antibody 23766 in MC38-tumor, B16-F10 tumor or LLC-OVA tumor bearing mice. Ex vivo γ-counting of the tumors from A) MC38-tumor bearing, C) B16-F10-tumor bearing or E) LLC-OVA-tumor bearing mice 90 min after injection with ^{99m}Tc-labeled single domain antibodies. Tumor-to-blood ratio of the different ^{99m}Tc-labeled single domain antibodies in B) MC38-tumor bearing mice, D) B16-F10-tumor bearing mice and F) LLC-OVA-tumor bearing mice, calculated by dividing the percentage activity per gram (% IA/g) in the tumor by the percentage of injected activity per gram in the blood. Biodistribution of the single domain antibodies in five mice is shown and expressed as mean±SD of percentage of injected activity per gram of organ or tissue (% IA/g). Statistical analysis was conducted using a two-way ANOVA test. ns, p>0.05; **, p<0.01; ***, p<0.001; ****, p<0.0001. [^{99m}Tc]-Tc-23766Nb: ^{99m}Tc-labeled CD163-specific single domain antibody 23766. [^{99m}Tc]Tc-Irr Nb: ^{99m}Tc-labeled irrelevant single domain antibody.

FIG. 8. Ex vivo flow cytometry data of the CD163-specific single domain antibody 23766 in MC38-tumor, B16-F10 tumor or LLC-OVA tumor bearing mice. CD163 expression was detected as mean fluorescent intensity (AMFI). The LLC-OVA tumor model shows the highest CD163 expression on all types of macrophages validating the highest uptake of the anti-CD163 immunotracer in this tumor model. Data presented as mean±S.D.

FIG. 9. Determination of single domain antibody binding affinities after NOTA-conjugation via ELISA and a cell-binding study. (A) Binding of different concentrations of the ⁶⁸Gallium-labeled anti-CD163 single domain antibody (“[⁶⁸Ga]Ga-NOTA-anti-CD163 Nb”) to the human CD163-Avi-Hexahistine protein, compared to binding of a control irrelevant ⁶⁸Gallium-labeled single domain antibody (“[⁶⁸Ga]Ga-NOTA-Irr Nb”) (B) Single domain antibody binding of different concentrations of the ⁶⁷Gallium-labeled anti-CD163 single domain antibody to HEK293T cells overexpressing mouse CD163.

FIG. 10. The ⁶⁸Gallium-labeled anti-CD163 immunotracer shows specific in vivo binding to CD163⁺ cells. A-B) Representative μPET/CT images of CD163 knock-out (CD163^{-/-}) and wild-type (WT) mice intravenously injected with the (A) ⁶⁸Gallium-labeled irrelevant single domain antibody (“[⁶⁸Ga]Ga-NOTA-Irr Nb”) and (B) ⁶⁸Gallium-labeled anti-CD163 immunotracer (“[⁶⁸Ga]Ga-NOTA-anti-CD163 Nb”). Cervical lymph nodes (upper arrow), liver (middle arrow) and bone marrow (lower arrow) are highlighted in wild-type (WT) mice. (C-F) Ex vivo uptake values of anti-CD163 immunotracer (“aCD163”) or irrelevant immunotracer (“Irr”) are shown in liver, spleen, cervical lymph nodes, and bone marrow in CD163 knock-out (CD163^{-/-}) and wild-type (WT) mice. All data are plotted as mean±S.D. of percentage of injected activity per gram of organ or tissue (% IA/g). Statistical analysis was performed using an unpaired two-tailed t-test. **, p<0.01; ***, p<0.001; ****, p<0.0001.

FIG. 11. The ⁶⁸Gallium-labeled anti-CD163 immunotracer is able to visualize macrophage distribution during anti-macrophage therapy. A) Individual growth curves of PLX-treated LLC-OVA tumor bearing mice showing responders (R) with a reduced tumor growth, partial responders (PR) with a partial reduction of tumor growth and non-responders (NR) showing unaffected tumor growth. B-C) Representative whole-body μPET/CT images of (B) untreated mice and (C) macrophage depleted mice (non-responder (NR) and responder (R)) intravenously injected with the ⁶⁸Ga-labeled anti-CD163 immunotracer at the last day of the therapy experiment. No or very little signal uptake is seen in the lymph nodes (upper arrow), bone marrow (middle arrow) and liver (lower arrow) of mice that were depleted and single domain antibody uptake in the tumor (lower arrow) is also lower as compared to untreated mice. Tumors are delineated by a dotted line. D-E) Correlation plots showing a significant correlation between (D) % IA in the tumor and tumor volume and (E) percentage of CD163⁺ MHC-II^{low} F4/80 expressing macrophages and tumor volume. F-G) Graphs representing flow cytometry results of (F) ratio of the percentage of MHC-II^{high}/MHC-II^{low} cells as part of the live cells and (G) percentage of CD163⁺ cells as part of the macrophages. PLX: macrophage-depleting compound PLX3397.

### DETAILED DESCRIPTION TO THE INVENTION

For purposes of diagnostic or molecular imaging in vivo, the imaging agent must be able to arrive at its target with high efficiency. This requires a combination of small-enough size in order to be able to achieve sufficient tissue penetration, selective binding to the target in order to achieve a high signal/noise ratio at the target site, and low overall body retention or accumulation (as a consequence of elimination from the body; typically in liver or kidneys) to avoid sites of high background signal which negatively influence signals at the target site.

In work leading to the current invention, first of all an immunoglobulin single variable domain (ISVD) molecule, herein also referred to a single domain antibody (sdAb), binding with high specificity to human and murine CD163 (hCD163 and mCD163, respectively) was identified.

The anti-CD163 sdAb was identified after screening of llama immune libraries and was evaluated for binding (on CD163-expressing cells) and affinity using enzyme-linked immunosorbent assay (ELISA), flow cytometry and Surface Plasmon Resonance (SPR). Single photon emission computed tomography imaging in mice following intravenous injection of Technetium-99m (^{99m}Tc)-labelled, ⁶⁸Ga-labelled or ⁶⁷Ga-labelled anti-CD163 sdAb revealed that this sdAb has several properties to make it an interesting diagnostic: (i) cross-reactivity for human and murine CD163; (ii) good in vivo visualization of macrophage rich organs (lymph nodes, liver, intestines, bone marrow) in healthy mice (compared to an irrelevant ISVD) and (ii) as well as discriminating between presence or absence of uptake of CD163+ macrophages in tumors, with a tumor-to-blood ratio significantly higher compared to an irrelevant ISVD. Surprisingly, compared to a CD206 sdAb-based immunotracer, the CD163 sdAb-based immunotracer displays a higher macrophage specificity resulting in lower background in liver tissue. These combined characteristics render the identified CD163-binding ISVD well-suited as diagnostic agent, e.g. for molecular imaging.

Based hereon, the invention is defined in the following aspects and embodiments, and described in more detail hereafter. As the invention relates to polypeptides comprising complementarity determining regions (CDRs), some explanation is first provided on how such CDRs are determined.

The determination of the CDR regions in an antibody/immunoglobulin sequence generally depends on the algorithm/methodology applied (Kabat-, Chothia-, Martin (enhanced Chothia), IMGT (ImMunoGeneTics information system)-numbering schemes; see, e.g. http://www.bioinf.org.Uk/abs/index.html#kabatnum and http://www.imgt.org/IMGTScientificChart/Numbering/IMGTnumbering.html). Applying different methods to the same antibody/immunoglobulin sequence may give rise to different CDR amino acid sequences wherein the differences may reside in CDR sequence length and/or -delineation within the antibody/immunoglobulin/I(S) VD sequence. The CDRs of the CD163-binding polypeptides of the invention can therefore be described as the CDR sequences as present in the single variable domain anti-CD163 antibody characterized herein, or alternatively as determined or delineated according to a well-known methodology such as according to the Kabat-, Chothia-, Martin (enhanced Chothia), or IMGT-numbering scheme or -method. The CDR sequences defined in SEQ ID NOs: 2-4, for instance, have, been delineated from the CD163 single domain antibody defined by SEQ ID NO:1 by means of the Kabat method. Applying another method may result in CDR sequences (slightly) different from those defined in SEQ ID NOs: 2-4 (the FR sequences, see further, then differ accordingly).

In a first aspect, the invention relates to polypeptides specifically binding to human and murine CD163 (also referred to herein as polypeptides specifically binding to CD163 or CD163-binding polypeptides), wherein the amino acid sequence of the polypeptide is comprising a CDR1 region, a CDR2 region, and a CDR3 region, wherein the CDR1, CDR2 and CDR3 regions are selected from those CDR1, CDR2 and CDR3 regions, respectively, as present in the CD163-binding single domain antibody or immunoglobulin variable domain (IVD) or immunoglobulin single variable domain (ISVD) defined by SEQ ID NO:1.

In particular, the polypeptides specifically binding to human and murine CD163 comprise an immunoglobulin (single) variable domain conveying specificity of the polypeptide for binding to human and murine CD163 wherein the I(S) VD is comprising a CDR1 region, a CDR2 region, and a CDR3 region, wherein the CDR1, CDR2 and CDR3 regions are the CDR1, CDR2 and CDR3 regions, respectively, as present in the CD163-binding single domain antibody defined by SEQ ID NO:1.

In an embodiment thereto, the CDR regions are determined by applying the Kabat, Chothia, Martin, or IMTG method to SEQ ID NO:1. In a more specific embodiment, the CDR regions are determined by the Kabat method and further defined as a CDR1 region defined by SEQ ID NO:2; a CDR2 region defined by SEQ ID NO:3; and a CDR3 region defined by SEQ ID NO:4.

In a further embodiment, polypeptides specifically binding to human and murine CD163, such as polypeptides specifically binding to human and murine CD163 comprising a CD163-specific immunoglobulin (single) variable domain (I(S) VD), are characterized by further comprising at least a framework region (FR) such as a framework region from an immunoglobulin (single) variable domain, wherein the I(S) VD polypeptide can comprise up to 4 FR regions (FR1 preceding CDR1; FR2 interspersed between CDR1 and CDR2; FR3 interspersed between CDR2 and CDR3; FR4 following CDR3; wherein the relative positioning referred to is from the amino- to carboxy-terminus of the I(S) VD). In particular, FR1 regions can be defined by SEQ ID NO:5. FR2 regions can be defined by SEQ ID NO:6. FR3 regions can be defined by SEQ ID NO:7. And FR4 region can be defined by SEQ ID NO:8. Herein the sequence-defined FR regions are delineated based on the delineation of the respective CDR regions as determined according to the Kabat method; these FR regions thus can slightly differ in case the CDR regions are determined according to a non-Kabat method.

In any of the above relating to the FR regions, a lysine can be changed into alanine which is useful for conjugation to NOTA-chelator (see further) or other imaging moieties; such substitution obviously is on the condition that binding affinity of the CD163-binding polypeptide to CD163 is not significantly affected.

In a further embodiment, any of the above polypeptides specifically binding to human and murine CD163, such as any of the above polypeptides specifically binding to human and murine CD163 comprising a CD163-specific immunoglobulin (single) variable domain, are characterized by further comprising a moiety extending the half-life of the polypeptide once administered to a subject. Such half-life extending moiety can for instance be a serum albumin binding I(S) VD, or albumin itself. Other half-life extension modalities include PEGylation (or any modification such as glycol-PEGylation, biotinylated PEG), attaching (whether or not in the form of a fusion protein) peptides such as XTEN, PAS (“Pro Ala Ser”), ELP (elastin-like polypeptide), GLK (gelatin-like protein), HAPylation (adding (Gly4Ser)n peptide), and adding a polysaccharide moiety (reviewed in e.g. Zaman et al. 2019, J Controlled Release 301:176-189).

In any of the above, the CDR regions and/or FR regions and/or the I(S) VD may be humanized. Humanized CDRs and/or FRs and/or I(S) VDs can be obtained in any suitable manner known and thus are not strictly limited to polypeptides that have been obtained using a polypeptide that comprises a naturally occurring VHH domain as starting material. Humanized immunoglobulin single variable domains, may have several advantages, such as a reduced immunogenicity, compared to the corresponding naturally occurring VHH domains. Such humanization generally involves replacing one or more amino acid residues in the sequence of a naturally occurring CDR and/or framework region (FR) with the amino acid residues that occur at the same position in a human VH domain, such as a human VH3 domain. The humanizing substitutions should be chosen such that the resulting humanized immunoglobulin domains still retain the favourable properties of the originator immunoglobulin (or further improved by e.g. affinity maturation). The skilled person will be able to select humanizing substitutions or suitable combinations of humanizing substitutions, which optimize or achieve a suitable balance between the favourable properties provided by the humanizing substitutions on the one hand and the favourable properties of naturally occurring VHH domains on the other hand. In general, the specificity of binding to the target is not significantly (negatively) affected in a humanized antibody/immunoglobulin/I(S) VD (or polypeptide comprising such antibody/immunoglobulin/I(S) VD) and, in general, the affinity and/or avidity of binding to the target is not significantly (negatively) affected in a humanized antibody/immunoglobulin/I(S) VD (or polypeptide comprising such antibody/immunoglobulin/I(S) VD).

The CD163-binding polypeptides of the invention may comprise (in a fusion, conjugated therewith, or complexed therewith), one or more non-(poly) peptidic constituents such as detectable moieties (see further) or such as being pegylated (e.g. WO2017/059397), one or more further polypeptide(s) or polypeptide domain(s) such as e.g. a His-tag, or a peptide ligase motif such as a sortag motif (sortase peptide ligase amino acid substrate motif LPXTG (SEQ ID NO:9), e.g. LPETG (SEQ ID NO:10); Mao et al. 2004, J Am Chem Soc 126:2670-2671) or a peptide asparaginyl ligases motif (recognized by e.g. butelase 1 or VyPAL2; motif sequence being NXL wherein X can be e.g. Gly, Ser, Ala, Gln; Zhang et al. 2022, Int J Mol Sci 23:458; Hu et al. 2022, Plant Cell 34:4936-4949). Such moieties/tags/motifs are referred to herein as “functional moiety”. In one instance, the CD163-binding polypeptide or CD163-specific I(S) VD itself may be duplicated or multiplicated (wherein the monomers are e.g. connected through a flexible linker such as a linker based on Gly-Pro repeats, Pro-Ala repeats, Gly-Ser repeats, or combinations thereof) to form a multivalent (though monospecific) binding molecule. In another instance, the further polypeptide or polypeptide domain (which may be connected through a flexible linker such as a linker based on Gly-Pro repeats, Pro-Ala repeats, Gly-Ser repeats, or combinations thereof, to the CD163-binding polypeptide; or included in the CD163 binding polypeptide as a fusion protein) may confer increased serum half-life (e.g. a serum albumin binding protein or peptide; see above).

Thus, in any of the above, the CD163 binding polypeptide may further comprise a functional moiety. In one embodiment, the functional moiety is a detectable moiety. CD163-binding polypeptides as defined herein and carrying a detectable moiety therewith may be immunotracers; in case the detectable moiety is a radiolabel, the CD163 binding polypeptides may be radioimmunotracers.

Both bare CD163-binding polypeptides (not comprising a detectable moiety) as described hereinabove and CD163-binding polypeptides comprising a detectable moiety are useful when envisaging the in vivo imaging application. Indeed, bare CD163-binding polypeptides may be co-administered with CD163-binding polypeptides comprising a detectable moiety to a subject, or may be administered to a subject prior to administering CD163-binding polypeptides comprising a detectable moiety, in order to mask the sink(s) of the CD163-binding polypeptides, more in particular the kidney sink; as such, sink background signals can be reduced. Moreover, it has been reported that preloading of unlabeled antibody may prolong the imaging window of the labeled antibodies (Nishio et al. 2020, Mol Imaging Biol 22:156-164).

A “detectable moiety” in general refers to a moiety that emits a signal or is capable of emitting a signal upon adequate stimulation, and is detectable by any means, preferably by a non-invasive means, once inside the human body. Furthermore, the detectable moiety may allow for computerized composition of an image, as such the detectable moiety may be called an imaging agent. Detectable moieties include fluorescence emitters, positron emitters, radioemitters, etc.

Measuring the amount of detectable moiety/imaging agent (comprised in, carried by, coupled to, chelated on a CD163-binding polypeptide) is typically done with a device counting radioactivity or determining radiation (which can be of photonic nature) density or radiation concentration. The counted or determined radioactivity can be transformed into an image. Depending on the nature of the emission by the detectable moiety, it may be detectable by techniques such as PET (positron emission tomography), SPECT (single-photon emission computed tomography), fluorescence imaging, fluorescence tomography, near infrared imaging, near infrared tomography, optical tomography, etc. Examples of radioemitters/radiolabels include ⁶⁸Ga, ^{110m}In, ¹⁸F, ⁴⁵Ti, ⁴⁴Sc, ⁴⁷Sc, ⁶¹Cu, ⁶⁰Cu, ⁶²Cu, ⁶⁶Ga, ⁶⁴Cu, ⁵⁵Ca, ⁷²As, ⁸⁶Y, ⁹⁰Y, ⁸⁹Zr, ¹²⁵I, ⁷⁴Br, ⁷⁵Br, ⁷⁶Br, ⁷⁷Br, ⁷⁸Br, ¹¹¹In, ^{114m}In, ¹¹⁴In, ^{99m}Tc, ¹¹C, ³²Cl, ³³Cl, ³⁴Cl, ¹²³I, ¹²⁴I, ¹³¹I, ¹⁸⁶Re, ¹⁸⁸Re, ¹⁷⁷Lu, ⁹⁹Tc, ²¹²Bi, ²¹³Bi, ²¹²Pb, ²²⁵Ac, ¹⁵³Sm, and ⁶⁷Ga. Fluorescence emitters include cyanine dyes (e.g. Cy5, Cy5.5, Cy7, Cy7.5), indolenine-based dyes, benzoindolenine-based dyes, phenoxazines, BODIPY dyes, rhodamines, Si-rhodamines, Alexa dyes, and derivatives of any thereof.

Many of the radionuclides have a metallic nature and are typically incapable of forming stable covalent bonds with proteins or peptides. One solution is to label proteins or peptides with radioactive metals by means of chelators, i.e. multidentate ligands, which form non-covalent compounds, called chelates, with the metal ions. A CD163 binding polypeptide may thus be coupled in any way to such chelator, which enables incorporation of a radionuclide; this allows a radionuclide to be coordinated, chelated or complexed to the CD163-binding polypeptide. Chelators include polyaminopolycarboxylate-type chelators which can be macrocyclic or acyclic. A polyaminopolycarboxylate chelator can be conjugated to a CD163-binding polypeptide e.g. via a thiol group of a cysteine residue or via an epsilon amine group of a lysine residue. Macrocyclic chelators for radioisotopes such as indium, gallium, yttrium, bismuth, radioactinides and radiolanthanides include DOTA (1,4,7,10-tetraazacyclododecane-1,4,7,10-tetraacetic acid) and derivatives thereof such as maleimidomonoamide-DOTA (1,4,7,10-tetraazacyclododecane-1,4,7-tris-acetic acid-10-maleimidoethylacetamide), DOTAGA (2,2′,2″-(10-(2,6-dioxotetrahydro-2H-pyran-3-yl)-1,4,7,10-tetraazacyclododecane-1,4,7-triyl)triacetic acid) with said polypeptide. Other chelators include NOTA (1,4,7-triazacyclononane-1,4,7-triacetic acid), and derivatives thereof such as -carboxy-4-((2,5-dioxopyrrolidin-1-yl)oxy)-4-oxobutyl)-1,4,7-triazonane-1,4-NODAGA (2,2′-(7-(1 di-yl)diacetic acid). Acyclic polyaminopolycarboxylate chelators include different derivatives of DTPA (diethylenetriamine-pentaacetic acid). Further chelating agents include DFO, CB-DO2A, 3p-C-DEPA, TCMC, Oxo-DO3A, TE2A, CB-TE2A, CB-TE1A1P, CB-TE2P, MM-TE2A, DM-TE2A, diamsar, NODASA, NETA, TACN-TM, 1B4M-DTPA, CHX-A″-DTPA, TRAP, NOPO, AAZTA, DATA, H2dedpa, H4octapa, H2azapa, H5decapa, H6phospa, HBED, SHBED, BPCA, CP256, PCTA, HEHA, PEPA, EDTA, TETA, and TRITA.

The detectable moiety in a CD163-binding polypeptide, may itself be comprised in a prosthetic group and the prosthetic group may be linked to the polypeptide through a chelator or conjugating moiety such as a cyclooctyne comprising a reactive group that forms a covalent bond with an amine, carboxyl, carbonyl or thiol functional group on the CD163-binding polypeptide. Cyclooctynes include dibenzocyclooctyne (DIBO), biarylazacyclooctynone (BARAC), dimethoxyazacyclooctyne (DIMAC) and dibenzocyclooctyne (DBCO), DBCO-PEG4-NHS-Ester, DBCO-Sulfo-NHS-Ester, DBCO-PEG4-Acid, DBCO-PEG4-Amine or DBCO-PEG4-Maleimide. An example of an ¹⁸F-labelled prosthetic group is ¹⁸F-3-(2-(2-(2-(2-azidoethoxy) ethoxy) ethoxy) ethoxy)-2-fluoropyridine (¹⁸F-FFPEGA). Other ¹⁸F-labelled prosthetic groups include N-Succinimidyl-4-[¹⁸F]fluorobenzoate ([¹⁸F]SFB) (e.g. Li et al. 2014, Applied Radiation and Isotopes 94:113-117); I-labelled prosthetic groups include N-succinimidyl 4-guanidinomethyl-3-[(*)I] iodobenzoate ([(*)I]SGMIB) and N-succinimidyl 3-guanidinomethyl-5-[(*)I]iodobenzoate (iso-[(*)I]SGMIB) wherein (*)I is for instance 131I (see e.g. Choi et al. 2014, Nucl Med Biol 41:802-812).

Conjugation methods as described above may result in heterogeneous tracer populations. Site-specific conjugation strategies try to overcome this shortcoming and include chemoenzymatic methods to couple polypeptides such as antibodies/immunoglobulins/I(S) VDs with a chelator or detectable moiety such as via sortase-mediated transpeptidation (Antos et al. 2009, Curr Protoc Protein Sci, Chapter 15: unti-15.3) (reviewed by e.g. Massa et al. 2016, Exp Opin Drug Deliv 13:1149-1163) or peptide ligase-mediated conjugation (see above). The CD163-binding polypeptides as described hereinabove thus may have the detectable moiety linked to a specific site comprised in the polypeptide, such as to form a homogeneous or quasi homogeneous population of tracer molecules.

Other aspects relate to isolated nucleic acids encoding a CD163-binding polypeptide as described hereinabove; to vectors comprising such nucleic acid; and to host cells comprising such nucleic acid or vector, and/or expressing CD163-binding polypeptide as described hereinabove.

A further aspect relates to pharmaceutical compositions comprising a CD163-binding polypeptide as described hereinabove (CD163-binding polypeptides without/not comprising a functional moiety, CD163-binding polypeptides with/comprising a functional moiety, or CD163-binding polypeptides with/comprising a detectable moiety). Such pharmaceutical compositions comprise a CD163-binding polypeptide as described hereinabove formulated in a suitable excipient. The suitable excipient is compatible with administration to a subject, e.g. is not toxic. On the other hand, the excipient may function in e.g. stabilizing or solubilizing the CD163-binding polypeptide such as with/comprising a functional moiety.

Yet a further aspect relates to CD163 binding polypeptides as described hereinabove, or to a pharmaceutical composition comprising them for use in diagnosis, for use in surgery or in guiding surgery, for use in therapy monitoring, and in particular for use as an imaging agent such as described herein. Alternatively, the invention relates to methods of diagnosis or therapy monitoring, said methods comprising administration of a CD163 binding polypeptide as described hereinabove, or of a pharmaceutical composition comprising it, to a subject. As a result of the administration, the presence of CD163+ cells can be diagnosed or the fluctuation of such cells before, after start or during a therapy such as an immunomodulating therapy can be followed up. Alternatively, the invention relates to methods of surgical resection of a tumor, said methods comprising administration of a CD163 binding polypeptide as described hereinabove, or of a pharmaceutical composition comprising it, to a subject, wherein the CD163 binding polypeptide, especially when comprising a detectable moiety, can assist in delineating the tumor during resection. In a particular embodiment, the CD163 binding polypeptides as described hereinabove are applied in the field of cancer or tumor imaging, in the field of monitoring of cancer or tumor therapy, in the field of cancer or tumor diagnosis, or in the field of cancer or tumor surgery or guiding cancer or tumor surgery.

Specificity or selectivity of cell targeting, in particular macrophage cell targeting, refers to the situation in which a composition, at a certain concentration, is interacting (such as binding) with the intended target cell with higher efficacy (e.g. with an at least 2-fold, 5-fold, or 10-fold higher efficacy, or e.g. with at least 20-, 50- or 100-fold higher efficacy) than the efficacy with which the composition is interacting with other cells (not intended as target cell). Exclusivity of cell targeting refers to the situation in which a composition is interacting only with the intended target cell.

### Diagnosis

In general “diagnosis” herein refers to detection of human or murine CD163 or of cells displaying human or murine CD163. This can be ex vivo or in vitro such as in a sample from a (human) subject (and such as by for instance ELISA, immunocytochemistry (ICH), western blot, or surface Plasmon resonance). This can also be in vivo diagnosis, in particular non-invasive in vivo diagnosis such as by medical imaging or molecular imaging as described hereinabove. Diagnosis, whether on a sample from a (human) subject or by in vivo (imaging) methods allows to monitor response to therapy, such as response to immunotherapy or an immunomodulating therapy, such as therapy of a subject having a tumor or having cancer. Diagnosis, and especially imaging, may also assist in defining e.g. a tumour in need of surgical resection, thus in assisting surgery or guiding surgery.

### Therapy Monitoring

As examples of immunomodulating therapeutic compounds, the FDA has approval anti-PD-1 mAbs pembrolizumab, nivolumab and cemiplimab; anti-PD-L1 mAbs durvalumab, atezolizumab and avelumab; anti-CTLA4 mAb ipilimumab; and the combination of anti-LAG3 mAb relatlimab and nivolumab, which have since become available as standard-of-care for several cancer types. The downside of this success story is the high cost of such treatments, easily surpassing $100,000 per patient (e.g. Aguiar et al. 2017, Ann Oncol 28:2256-2263), and the observation that these immune checkpoint blockers are only of benefit for a subset of patients (e.g. Alsaab et al. 2017, Front Pharmacol 8:561). The failure rate, combined with the high cost for society, drives the search for predictive biomarkers that can help select the right treatment for the right patient. Currently the most commonly used predictive biomarker is PD-L1 expression assessed via IHC on tumor biopsies, although limitations are obviously present. Limitations such as heterogeneous expression, the role of expression outside of the tumor, and its dynamic expression during the disease process. Such limitations could be overcome by noninvasive molecular imaging using radiolabeled tracers that allow deep tumor penetration and repeated quantification of a reliable marker—this would enable mapping of primary tumors and metastatic lesions or of the immune landscape within such tumors or lesions both before and during the treatment.

Immunotracer-based tumor imaging in vivo can assist in disease diagnostics, patient stratification (determining which patients are more likely to respond to immunotherapy), disease monitoring (changes in the tumor images obtained during therapy reflect response or non-response to immunotherapy) and the design and development of new immunotherapies (throughout pre-clinical or clinical development). In particular, imaging (such as immunoPET imaging) of immune cells, in particular CD163+ immune cells, based on labeled anti-CD163 moieties of the current invention can likewise assist in monitoring the efficacy of immunotherapy, immunogenic or immunomodulating therapy, while also assisting in patient stratification and providing valuable information when designing and/or developing new immunotherapies, immunogenic therapies or immunomodulating therapies.

### Immunotherapy and Immunogenic Therapy

Immunotherapy in general is defined as a treatment that uses the body's own immune system to help fight a disease, more specifically cancer in the context of the current invention. Immunotherapeutic treatment as used herein refers to the reactivation and/or stimulation and/or reconstitution of the immune response of a mammal towards a condition such as a tumour, cancer or neoplasm evading and/or escaping and/or suppressing normal immune surveillance. The reactivation and/or stimulation and/or reconstitution of the immune response of a mammal in turn in part results in an increase in elimination of tumorous, cancerous or neoplastic cells by the mammal's immune system (anticancer, antitumour or anti-neoplasm immune response; adaptive immune response to the tumour, cancer or neoplasm). Immunotherapeutic agents of particular interest include immune checkpoint inhibitors (such as anti-PD-1, anti-PD-L1 or anti-CTLA-4 antibodies), bispecific antibodies bridging a cancer cell and an immune cell, dendritic cell vaccines, oncolytic viruses, cell-based therapies (e.g. CAR-T). Immunotherapy is a promising new area of cancer therapeutics and several immunotherapies are being evaluated pre-clinically as well as in clinical trials and have demonstrated promising activity (Callahan et al. 2013, J Leukoc Biol 94:41-53; Page et al. 2014, Annu Rev Med 65:185-202). However, not all the patients are sensitive to immune checkpoint blockade and sometimes PD-1 or PD-L1 blocking antibodies accelerate tumour progression. An overview of clinical developments in the field of immune checkpoint therapy is given by Fan et al. 2019 (Oncology Reports 41:3-14). Monoclonal antibodies targeting and inhibiting PD-1 include pembrolizumab, nivolumab, and cemiplimab. Monoclonal antibodies targeting and inhibiting PD-L1 include atezolizumab, avelumab, and durvalumab. Monoclonal antibodies targeting and inhibiting CTLA-4 include ipilimumab. Combinatorial cancer treatments that include chemotherapies can achieve higher rates of disease control by impinging on distinct elements of tumour biology to obtain synergistic antitumour effects. It is now accepted that certain chemotherapies can increase tumour immunity by inducing immunogenic cell death and by promoting escape in cancer immunoediting, such therapies are therefore called immunogenic therapies as they provoke an immunogenic response. Drug moieties known to induce immunogenic cell death include bleomycin, bortezomib, cyclophosphamide, doxorubicin, epirubicin, idarubicin, mafosfamide, mitoxantrone, oxaliplatin, and patupilone (Bezu et al. 2015, Front Immunol 6:187). Other forms of immunotherapy include chimeric antigen receptor (CAR) T-cell therapy in which allogeneic T-cells are adapted to recognize a tumour neo-antigen and oncolytic viruses preferentially infecting and killing cancer cells. Treatment with RNA, e.g. encoding MLKL, is a further means of provoking an immunogenic response (Van Hoecke et al. 2018, Nat Commun 9:3417), as well as vaccination with neo-epitopes (Brennick et al. 2017, Immunotherapy 9:361-371).

### Applications of the CD163 Single Domain Antibodies

A number of applications related to targeting CD163 have been described in the art. For instance: U.S. Pat. No. 9,724,426B2 claims agents combining a CD163 binding moiety and a cytotoxic moiety or a drug, allowing the agent to be internalized in a cell when binding to a cell exposing CD163 on its surface. WO2011039510 refers to a CD163-binding molecule linked to an immunostimulatory agent which can e.g. be a Toll-like receptor (TLR) ligand. U.S. Pat. No. 9,476,890B2 claims a CD163 binding antibody linked to a prophylactic or therapeutic medicament. WO2017158436 refers to a fusion protein of an immunostimulatory agent and a targeting unit guiding the immunostimulatory agent to a tumor-associated macrophage. The targeting unit can be one binding to e.g. CD163 and can be e.g. an immunoglobulin single variable domain. US2018236076 refers to anti-CD25 antibodies (such as lacking an Fc receptor region) coupled to a photon absorber (IR-700) wherein the antibodies are targeting e.g. CD25+ cells (e.g. Tregs), which then can be selectively eliminated by photoimmunotherapy. This concept was theoretically expanded to, amongst other, CD163+ cells. WO2018156725 refers to tumor treatment with an antibody (or antigen binding fragment) conjugated to a cytotoxic compound wherein the antibody is binding to CD163, CD204, or CD206. US20220073638 refers to methods of increasing CD8+ T-cell infiltration in a tumor by administering an anti-CD163 antibody; and to methods of treating cancer combining an anti-CD163 antibody and an immune checkpoint inhibitor, the anti-CD163 antibody can herein be an antibody-drug conjugate. U.S. Pat. No. 10,751,284 refers to tumor associated macrophage-targeting liposomes loaded with a cytotoxic agent with the liposomes being targeted to the macrophages by a CD163-binding antibody. A CD206 single domain antibody conjugated to the Toll-like receptor 7/8 agonist imidazoquinoline IMDQ has been reported to repolarize tumor-associated macrophages into a tumoricidal state, and to reduce tumor growth (Bolli et al. 2021, Adv Sci 2021:2004574).

The benefits of the CD163-binding polypeptides according to this invention extend equally well to such applications. Indeed, for purposes of diagnostic or molecular imaging in vivo as well as for therapeutic purposes, the imaging agent or therapeutic agent must be able to arrive at its target with high efficiency. This requires a combination of small-enough size in order to be able to achieve sufficient tissue penetration. Selective binding to the target in order to achieve a high signal/noise ratio at the target site (imaging agent) likewise contributes to the specificity of a therapeutic agent. The cell-selectivity profile of the CD163-binding polypeptides according to this invention is furthermore ideal in avoiding as much as possible unwanted side effects.

The invention therefore in a further aspect relates to CD163-binding polypeptides according to the invention coupled to any of a prophylactic or therapeutic drug cytotoxic moiety or drug, an immunostimulatory agent, an immunosuppressive agent, a Toll-like receptor agonist, to a photon absorber, to a liposome or to a nanoparticle; as well as to compositions, such a pharmaceutical compositions comprising such conjugated molecules. In one embodiment thereto, the CD163-binding polypeptide is the CD163-binding single domain antibody or CD163-binding immunoglobulin single variable domain as defined hereinabove, such as defined by the CDR regions comprised in it, or as defined by the CDR and FR regions comprised in it. Such conjugated molecules, or compositions comprising them, are in particular for use as a medicament or for use in the manufacture of a medicament; or, depending on their payload, for/for use in/or for use in a method of treating or inhibiting (progression of) cancer or a tumor, for/for use in/or for use in a method of treating or inhibiting (progression of) an inflammatory or autoimmune disease, for/for use in/or for use in a method of treating or inhibiting (progression of) an infectious disease. When for use in in treating or inhibiting (progression of) cancer or a tumor, this can be in combination with or as part of a combination treatment with a further anti-cancer or anti-tumor agent. Such further anti-cancer or anti-tumor agent can e.g. be an immune checkpoint inhibitor (see above under immunotherapy) or a cytotoxic drug (see hereinafter).

The CD163 binding polypeptides according to the invention can in general be conjugated to any prophylactic or therapeutic drug; in particular such prophylactic or therapeutic drug can be selected based on its efficacy when targeted to CD163-positive cells, in particular to macrophages. In some embodiments, the prophylactic or therapeutic drug is attached to the CD163 binding polypeptide by a spacer arm, the length of it designed to avoid or reduce potential steric hindrance. Alternatively, the prophylactic or therapeutic drug is loaded into a nanoparticle, liposome, lipid nanoparticle, etc., with the loaded nanoparticle or liposome being conjugated to a CD163 binding polypeptide according to the invention.

Some examples of prophylactic or therapeutic drugs include cytotoxic drugs (such as for (use in) treating cancer), immunostimulatory drugs (such as for (use in) treating cancer), immunosuppressive drugs (such as for (use in) treating an inflammatory or autoimmune disease) and antimicrobial drugs (such as for (use in) treating an infectious disease).

Examples of cytotoxic drugs or moieties include alkylating agents (e.g. cisplatin, carboplatin), antimetabolites (e.g. methotrexate, azathioprine), antimitotics (e.g. vincristine), topoisomerase inhibitors (e.g. doxorubicine, etoposide), and toxins (e.g. calicheamicin).

Immunosuppressive drug include anti-inflammatory drugs. Such drugs include: glucocorticoids (e.g. cortisone and derivatives thereof; prednisone and derivatives thereof; dexamethasone and derivatives thereof; triamcinolone and derivatives thereof; paramethasone; betamethasone; fluhydrocortisone; fluocinolone); methotrexate; cyclophosphamide; 6-mercaptopurin; cyclosporine; tacrolimus; mycophenolate mofetil; sirulimus; everolimus; non-steroidal anti-inflammatory drugs (NSAIDs, such as aspirin, ibuprofen); steroids (such as vitamin D); disease-modifying anti-rheumatic drugs (DMARDs, such as penicillamin, sulfasalazin, cyclosporine).

Immunosuppressive drugs can be used in the treatment of an inflammatory and/or an autoimmune condition or disorder. Inflammatory and autoimmune conditions or disorders include arthritic diseases (such as rheumatoid arthritis, spondylitis, osteoarthritis); chronic inflammatory bowel disease (IBD, such as Crohn's disease, ulcerative colitis); peridontitis; psoriasis; asthma; systemic lupus erythematosus; multiple sclerosis; autoimmune chronic inflammatory diseases; connective tissue disease; autoimmune liver disease (such as biliary cirrhosis); sepsis; hemophagocytic syndrome; liver disease; liver failure; hepatitis; atherosclerosis; diabetes; obesity; non-alcoholic fatty liver disease; nonalcoholic steatohepatitis (NASH); alcoholic steatohepatitis (ASH); acute alcoholic hepatitis; joint inflammation; inflammation-induced cartilage destruction; liver cirrhosis; organ transplantation; Idiopathic Thrombocytopenia Purpura (ITP); sarcoidosis, uveitis; HLA-B27 positive uveitis; acute uveitis; macrophage activation syndrome; giant cell arthritis.

Immunostimulatory drugs may be drugs capable of stimulating one or more anti-tumour activity of a macrophage. Immunostimulatory drugs include cytokines and interleukins (e.g. interleukin-2), Toll-like-receptor (TLR) agonists such as a TLR7/8 ligand or agonist (e.g. IMDQ, the imidazoquinoline variant 1-(4-(aminomethyl)benzyl)-2-butyl-1H-imidazo[4,5-c]quinolin-4-amine), bacterial polysaccharides, costimulatory ligands (such as 41bb, CD80, CD86).

Antimicrobial drug include: antibiotics, anti-tuberculosis antibiotics (such as isoniazide, ethambutol), anti-retroviral drugs (for example an inhibitor of reverse transcription (such as zidovudin) or a protease inhibitor (such as indinavir)), drugs with effect on leishmaniasis (such as Meglumine antimoniate). Antimicrobial drugs can be used in the treatment of a condition or disorder caused by an micro-organism such as tuberculosis, AIDS, HIV infection, Leishmaniasis.

The CD163 binding polypeptides according to the invention can in general be conjugated to e.g. photon absorbers such that the resulting CD163 binding polypeptide conjugate can be used in near-infrared photoimmunotherapy (NIR-PIT) upon activation of the photon absorber by near-infrared light. Photon absorbers include the photo-activatable silica-phthalocyanine dye (IRDye700DX).

In a final aspect, the invention relates to methods for producing a CD163-binding polypeptide according to the invention, such methods comprising the steps of:
expressing the CD163-binding polypeptide in a suitable host cell (such as comprising a nucleic acid or vector as described herein), or synthetic manufacture of the CD163-binding polypeptide; and
purifying the expressed or synthesized/manufactured CD163-binding polypeptide.

Such methods may further comprise a step of coupling, incorporating, binding, ligating, bonding, complexing, chelating, conjugating (e.g. site-specifically conjugating) or otherwise linking, covalently or non-covalently, a detectable moiety to the purified CD163-binding polypeptide, or a prophylactic or therapeutic drug cytotoxic moiety or drug, immunostimulatory agent, immunosuppressive agent, Toll-like receptor agonist, photon absorber, liposome or nanoparticle.

### Other Definitions

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

The term “defined by SEQ ID NO:X” as used herein refers to a biological sequence consisting of the sequence of amino acids or nucleotides given in the SEQ ID NO:X. For instance, a CDR defined in/by SEQ ID NO: X consists of the amino acid sequence given in SEQ ID NO:X. A further example is an amino acid sequence comprising SEQ ID NO:X, which refers to an amino acid sequence longer than the amino acid sequence given in SEQ ID NO:X but entirely comprising the amino acid sequence given in SEQ ID NO:X (wherein the amino acid sequence given in SEQ ID NO:X can be located N-terminally or C-terminally in the longer amino acid sequence, or can be embedded in the longer amino acid sequence), or to an amino acid sequence consisting of the amino acid sequence given in SEQ ID NO:X.

The term “antibody” as used herein, refers to an immunoglobulin (lg) molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The term “immunoglobulin domain” as used herein refers to a globular region of an antibody chain (such as e.g., a chain of a conventional 4-chain antibody or a chain of a heavy chain antibody), or to a polypeptide that essentially consists of such a globular region. Immunoglobulin domains are characterized in that they retain the immunoglobulin fold characteristic of antibody molecules, which consists of a two-layer sandwich of about seven antiparallel β-strands arranged in two β-sheets, optionally stabilized by a conserved disulphide bond. The specificity of an antibody/immunoglobulin/I(S) VD for an antigen is defined by the composition of the antigen-binding domains in the antibody/immunoglobulin/I(S) VD (usually one or more of the CDRs, the particular amino acids of the antibody/immunoglobulin/I(S) VD interacting with the antigen forming the paratope) and the composition of the antigen (the parts of the antigen interacting with the antibody/immunoglobulin/l(S) VD forming the epitope). Specificity of binding is understood to refer to a binding between an antibody/immunoglobulin/I(S) VD with a single target molecule or with a limited number of target molecules that (happen to) share an epitope recognized by the antibody/immunoglobulin/l(S) VD.

Affinity of an antibody/immunoglobulin/I(S) VD for its target is a measure for the strength of interaction between an epitope on the target (antigen) and an epitope/antigen binding site in the antibody/immunoglobulin/I(S) VD. It can be defined as:

$${K}_{A} = \frac{[ Ab - Ag ]}{[ Ab ] [ Ag ]}$$

Wherein KA is the affinity constant, [Ab] is the molar concentration of unoccupied binding sites on the antibody/immunoglobulin/I(S) VD, [Ag] is the molar concentration of unoccupied binding sites on the antigen, and [Ab-Ag] is the molar concentration of the antibody-antigen complex.

Avidity provides information on the overall strength of an antibody/immunoglobulin/I(S) VD-antigen complex, and generally depends on the above-described affinity, the valency of antibody/immunoglobulin/I(S) VD and of antigen, and the structural interaction of the binding partners. The term “immunoglobulin variable domain” (abbreviated as “I(S) VD”) as used herein means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Methods for delineating/confining a CDR in an antibody/immunoglobulin/I(S) VD have been described hereinabove.

The term “immunoglobulin single variable domain” (abbreviated as “ISVD”), equivalent to the term “single variable domain”, defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an gG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associated) immunoglobulin domains such as light and heavy chain variable domains, i.e., by a VH-VL pair of immunoglobulin domains, which jointly bind to an epitope of the respective antigen. In contrast, immunoglobulin single variable domains are capable of specifically binding to an epitope of the antigen without pairing with an additional immunoglobulin variable domain. The binding site of an immunoglobulin single variable domain is formed by a single VH/VHH or VL domain. Hence, the antigen binding site of an immunoglobulin single variable domain is formed by no more than three CDRs. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody® (as defined herein, and including but not limited to a VHH); other single variable domains, or any suitable fragment of any one thereof. In particular, the immunoglobulin single variable domain may be a Nanobody® (as defined herein) or a suitable fragment thereof. Note: Nanobody®, Nanobodies® and Nanoclone® are registered trademarks of Ablynx N.V. For a general description of Nanobodies®, reference is made to the further description below, as well as to the prior art cited herein, such as e.g. described in WO2008/020079.

“VHH domains”, also known as VHHs, VHH domains, VHH antibody fragments, and VHH antibodies, have originally been described as the antigen binding immunoglobulin (variable) domain of “heavy chain antibodies” (i.e., of “antibodies devoid of light chains”; Hamers-Casterman et al (1993) Nature 363:446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody®, reference is made to the review article by Muyldermans 2001 (Reviews in Molecular Biotechnology 74:277-302), as well as to the following patent applications, which are mentioned as general background art: WO 94/04678, WO 95/04079 and WO 96/34103; WO 94/25591, WO 99/37681, WO 00/40968, WO 00/43507, WO 00/65057, WO 01/40310, WO 01/44301, EP 1134231 and WO 02/48193; WO 97/49805, WO 01/21817, WO 03/035694, WO 03/054016 and WO 03/055527; WO 03/050531; WO 01/90190; WO 03/025020; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825. As described in these references, Nanobody® (in particular VHH sequences and partially humanized Nanobody®) can in particular be characterized by the presence of one or more “Hallmark residues” in one or more of the framework sequences. A further description of the Nanobody®, including humanization and/or camelization of Nanobody®, as well as other modifications, parts or fragments, derivatives or “Nanobody® fusions”, multivalent constructs (including some non-limiting examples of linker sequences) and different modifications to increase the half-life of the Nanobody® and their preparations can be found e.g. in WO 08/101985 and WO 08/142164.

“Domain antibodies”, also known as “dAbs” (the terms “Domain Antibodies” and “dAbs” being used as trademarks by the GlaxoSmithKline group of companies) have been described in e.g., EP 0368684, Ward et al. 1989 (Nature 341:544-546), Holt et al. 2003 (Trends in Biotechnology 21:484-490) and WO 03/002609 as well as for example WO 04/068820, WO 06/030220, WO 06/003388. Domain antibodies essentially correspond to the VH or VL domains of non-camelid mammalians, in particular human 4-chain antibodies. In order to bind an epitope as a single antigen binding domain, i.e., without being paired with a VL or VH domain, respectively, specific selection for such antigen binding properties is required, e.g. by using libraries of human single VH or VL domain sequences. Domain antibodies have, like VHHs, a molecular weight of approximately 13 to approximately 16 kDa and, if derived from fully human sequences, do not require humanization for e.g. therapeutic use in humans. It should also be noted that single variable domains can be derived from certain species of shark (for example, the so-called “IgNAR domains”, see for example WO 05/18629).

Immunoglobulin single variable domains such as Domain antibodies and Nanobody® (including VHH domains and humanized VHH domains), can be subjected to affinity maturation by introducing one or more alterations in the amino acid sequence of one or more CDRs, which alterations result in an improved affinity of the resulting immunoglobulin single variable domain for its respective antigen, as compared to the respective parent molecule. Affinity-matured immunoglobulin single variable domain molecules of the invention may be prepared by methods known in the art, for example, as described by Marks et al. 1992 (Biotechnology 10:779-783), Barbas et al. 1994 (Proc Natl Acad Sci USA 91:3809-3813), Shier et al. 1995 (Gene 169:147-155), Yelton et al. 1995 (Immunol 155:1994-2004), Jackson et al. 1995 (J Immunol 154:3310-3319), Hawkins et al. 1992 (J Mol Biol 226:889-896), Johnson and Hawkins (Affinity maturation of antibodies using phage display, Oxford University Press, 1996). The process of designing/selecting and/or preparing a polypeptide, starting from an immunoglobulin single variable domain such as a Domain antibody or a Nanobody®, is also referred to herein as “formatting” said immunoglobulin single variable domain; and an immunoglobulin single variable domain that is made part of a polypeptide is said to be “formatted” or to be “in the format of” said polypeptide. Examples of ways in which an immunoglobulin single variable domain can be formatted and examples of such formats for instance to avoid glycosylation will be clear to the skilled person based on the disclosure herein.

Immunoglobulin single variable domains such as Domain antibodies and Nanobody® (including VHH domains) can be subjected to humanization, i.e. increase the degree of sequence identity with the closest human germline sequence. In particular, humanized immunoglobulin single variable domains, such as Nanobody® (including VHH domains) may be immunoglobulin single variable domains that are as generally defined for in the previous paragraphs, but in which at least one amino acid residue is present (and in particular, at least one framework residue) that is and/or that corresponds to a humanizing substitution (as defined herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized.

A “serum albumin binding agent”, or “serum albumin binding polypeptide”, as used herein, is a protein-based agent capable of specific binding to serum albumin. In various embodiments, the serum albumin binding agent may bind to the full-length and/or mature forms and/or isoforms and/or splice variants and/or fragments and/or any other naturally occurring or synthetic analogues, variants or mutants of serum albumin. In various embodiments, the serum albumin binding agent of the invention may bind to any forms of serum albumin, including monomeric, dimeric, trimeric, tetrameric, heterodimeric, multimeric and associated forms. In an embodiment, the serum albumin binding agent binds to the monomeric form of serum albumin. In an embodiment, the present serum albumin binding polypeptide comprises immunoglobulin variable domain with an antigen binding site that comprises three complementarity determining regions (CDR1, CDR2 and CDR3). In an embodiment said antigen binding site recognizes one or more epitopes present on serum albumin. In various embodiments, the serum albumin binding agent comprises a full length antibody or fragments thereof. In an embodiment, the serum albumin binding agent comprises a single domain antibody or an immunoglobulin single variable domain (ISVD). In a specific embodiment, the serum albumin binding agent binds to serum albumin of rat (Uniprot P02770). In a specific embodiment, the serum albumin binding agent binds to serum albumin of mouse (Uniprot P07724). In a specific embodiment, the serum albumin binding agent binds to human serum albumin (Uniprot P02768).

The aspects and embodiments described above in general may comprise the administration of a CD163 binding polypeptide or pharmaceutical composition comprising it to a mammal in need thereof, i.e., harbouring a tumour, cancer or neoplasm in need of (non-invasive) medical imaging, diagnosis, surgery (or guiding surgery) or therapy monitoring. In general an effective amount of the CD163-binding polypeptide or pharmaceutical composition comprising it is administered to the mammal in need thereof in order to meet the desired effect. The effective amount will depend on many factors such as route of administration and will need to be determined on a case-by-case basis by the physician. “Administering” means any mode of contacting that results in interaction between an agent (a CD163-binding polypeptide as described herein) or composition comprising the agent (such as a medicament or pharmaceutical composition) and an object (e.g. cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

The term “effective amount” refers to the dosing regimen of the agent (a CD163-binding polypeptide as described herein) or composition comprising the agent (e.g. pharmaceutical composition). The effective amount will generally depend on and/or will need adjustment to the mode of contacting or administration. To obtain or maintain the effective amount, the agent or composition comprising the agent may be administered as a single dose or in multiple doses. The effective amount may further vary depending on the severity of the condition that needs to be diagnosed, imaged, or operated; this may depend on the overall health and physical condition of the mammal or patient and usually a doctor's or physician's assessment will be required to establish what is the effective amount. The effective amount may further be obtained by a combination of different types of contacting or administration.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims. The content of the documents cited herein are incorporated by reference.

### EXAMPLES

### Example 1. Anti-CD163 Single Domain Antibody Binding with High Affinity to Both the Human and Mouse CD163 Receptor

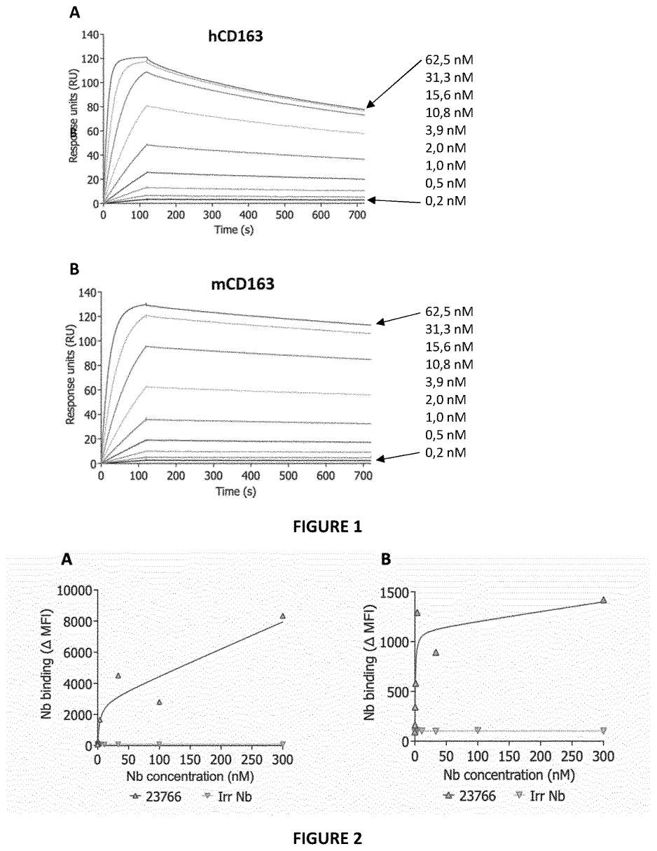

Two llamas were immunized with recombinant human CD163 (hCD163) protein and mouse CD163 (mCD163) protein to obtain cross-reactive CD163-specific single domain antibodies (sdAbs). Only 9 clones were considered cross-reactive after phage display panning and initial screening. After performing a ThermoFluor assay to determine the thermostability, a surface plasmon resonance (SPR) experiment analyzing the affinity towards both the recombinant hCD163 and the mCD163 protein and a flow cytometry experiment to test the affinity on HEK293T cells overexpressing hCD163 or mCD163, one lead cross-reactive CD163-targeting sdAb (sdAb 23766) was selected. This sdAb showed a high binding affinity towards both the hCD163 and the mCD163 protein via SPR (FIG. 1 and Table 1). Strong binding was also seen on HEK293T hCD163⁺ cells and HEK293T mCD163⁺ cells (FIG. 2 and Table 1) and the sdAb 23766 has a melting temperature (Tm) of 78.0° C. which means that this sdAb can be incubated at high temperatures (>50° C.) for radiolabeling (Tables 1A and 1B).

TABLE 1A
Overview of the in vitro characteristics of the cross-reactive lead CD163-specific sdAbs.

| | K_{D} hCD163 | K_{D} mCD163 | K_{D} HEK hCD163⁺ | K_{D} HEK mCD163⁺ | Melting |
|---|---|---|---|---|---|
| | protein | protein | cells (nM) | cells (nM) | temperature |
| sdAb | (nM) SPR | (nM) SPR | Flow cytometry | Flow cytometry | (° C.) |
| 23766 | 3.5 | 3.5 | 2.5 ± 2.5 | 2.0 ± 1.8 | 78.0 ± 0.0 |

TABLE 1B
Overview of affinity binding results determined via surface plasmon resonance
(SPR) and flow cytometry, and melting temperature determined via a thermostability
assay. Data presented as mean ± S.D of at least 3 independent experiments.

| K_{D} hCD163 | K_{D} mCD163 | K_{D} HEK hCD163⁺ | K_{D} HEK mCD163⁺ | Melting |
|---|---|---|---|---|
| protein | protein | cells (nM) | cells (nM) | temperature |
| (nM) SPR | (nM) SPR | Flow cytometry | Flow cytometry | (° C.) |
| 0.5 ± 0.3 | 0.4 ± 0.1 | 11.3 ± 16.4 | 3.5 ± 2.7 | 78.1 ± 0.03 |

The amino acid sequence of sdAb 23766 and its CDR and FR regions was determined. These sequences are depicted hereafter.

sdAb23766
(SEQ ID NO: 1)
DVQLVESGGG LVQPGGSLRL SCAASGITFS SYAVAWFRQA SGKEREFVAF IGWDGDTTYY
VDSVKGRFTI SRDNAKNMVY LQMNSLKPDD TAIYYCARHK TLWRSSWDNR PVQYDYWGQG
TQVTVSS
sdAb23766 CDR1:
(SEQ ID NO: 2)
GITFSSYA
sdAb23766 CDR2:
(SEQ ID NO: 3)
IGWDGDTT
sdAb23766 CDR3:
(SEQ ID NO: 4)
ARHKTLWRSSWDNRPVQYDY
sdAb23766 FR1:
(SEQ ID NO: 5)
DVQLVESGGGLVQPGGSLRLSCAAS
sdAb23766 FR2:
(SEQ ID NO: 6)
VAWFRQASGKEREFVAF
sdAb23766 FR3:
(SEQ ID NO: 7)
YYVDSVKGRFTISRDNAKNMVYLQMNSLKPDDTAIYYC
sdAb23766 FR4:
(SEQ ID NO: 8)
WGQGTQVTVSS

The human CD163 gene is located on chr12:7,470,811-7,503,893 (GRCh38/hg38; minus strand), alternatively on chr12:7,623,407-7,656,373 (GRCh37/hg19 by Entrez Gene; minus strand), alternately on chr12:7,623,409-7,656,489 (GRCh37/hg19 by Ensembl; minus strand). Reference mRNA sequences: GenBank accession nos. NM_001370145.1; NM_001370146.1; NM_004244.6; and NM_203416.4. A coding sequence for the human CD163 can further be found under e.g. GenBank accession no. DQ058615.1.

A coding sequence for the murine CD163 gene can be found under e.g. GenBank accession no. BC145793.1.

### Example 2. The Anti-CD163 Single Domain Antibody Specifically Targets Macrophages in Naïve and Tumor-Bearing Mice

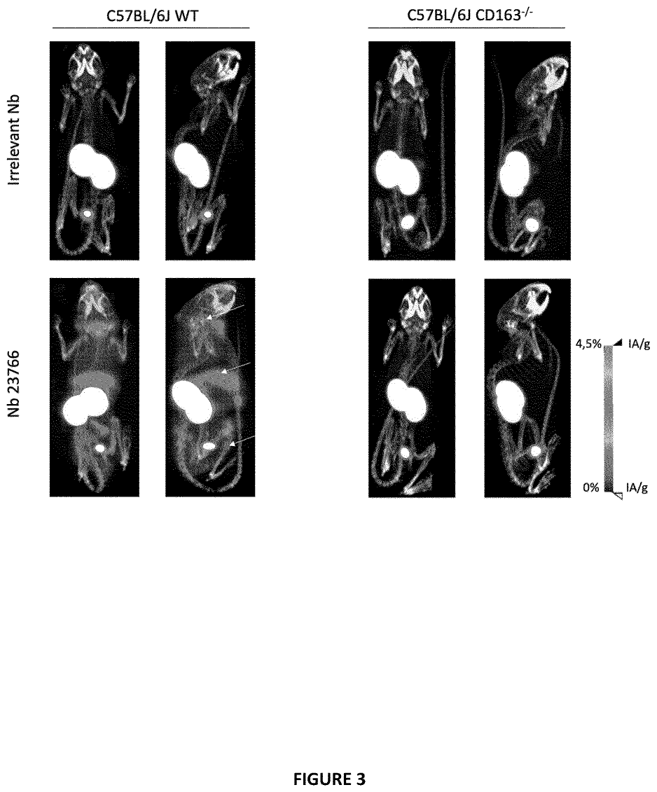

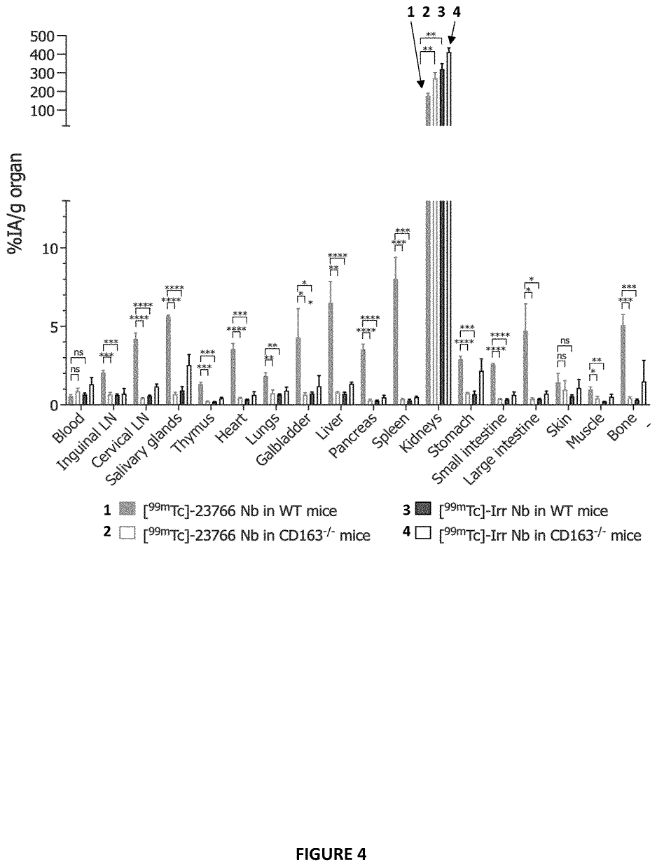

We next tested sdAb 23766 on selectivity, biodistribution, background signal and tracer accumulation in vivo. First, sdAb 23766 and the irrelevant sdAb R3b23 were site-specifically labeled with Technetium-99m (^{99m}Tc) via their C-terminal His-tag and injected intravenously in naïve C57BL/6J wild type (WT) mice (n=3) and C57BL/6J CD163 knock-out (^{-/-}) mice (n=3). One hour post injection, mice were imaged via a SPECT/CT camera, and their organs were harvested for ex vivo biodistribution analysis via gamma (v)-counting. The ^{99m}Tc-labeled sdAb 23766 showed high uptake in macrophage-rich organs such as cervical lymph nodes, liver, intestines, and bone marrow in naïve WT mice, while not showing uptake in CD163^{-/-} mice (FIG. 3). This implies that the signal uptake is specific for cells expressing the mCD163 receptor. No signal of the irrelevant sdAb is seen in WT nor CD163/mice. Specific uptake of the sdAb 23766 was confirmed via ex vivo γ-counting with also uptake seen in the spleen (FIG. 4). The splenic uptake was masked on the SPECT/CT images by the high signal in the kidneys, since sdAbs are cleared via the renal system but reabsorbed by the proximal tubule cells and retained in the renal cortex (Chigoho et al. 2021, Curr Opin Chem Biol 63:219-228).

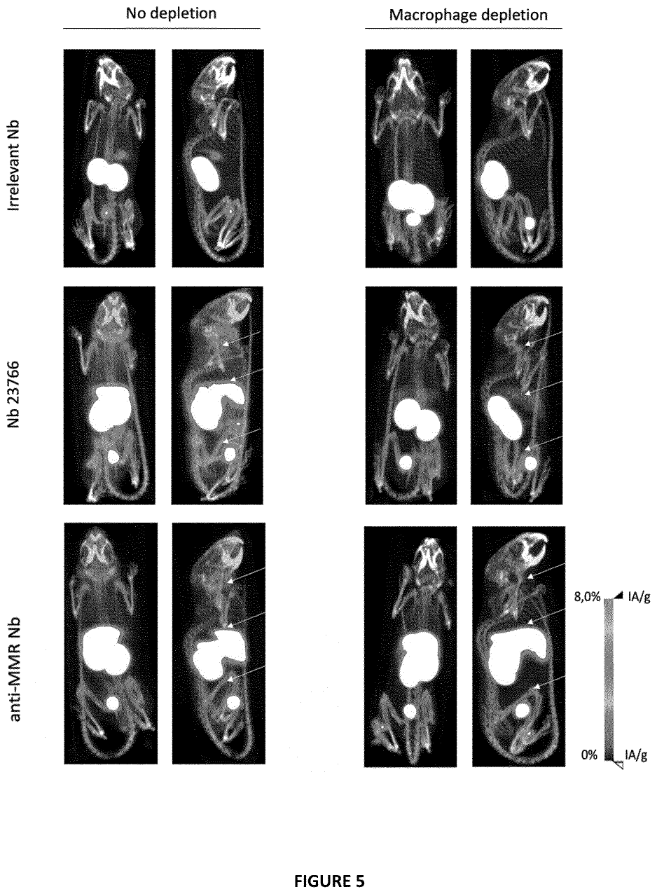

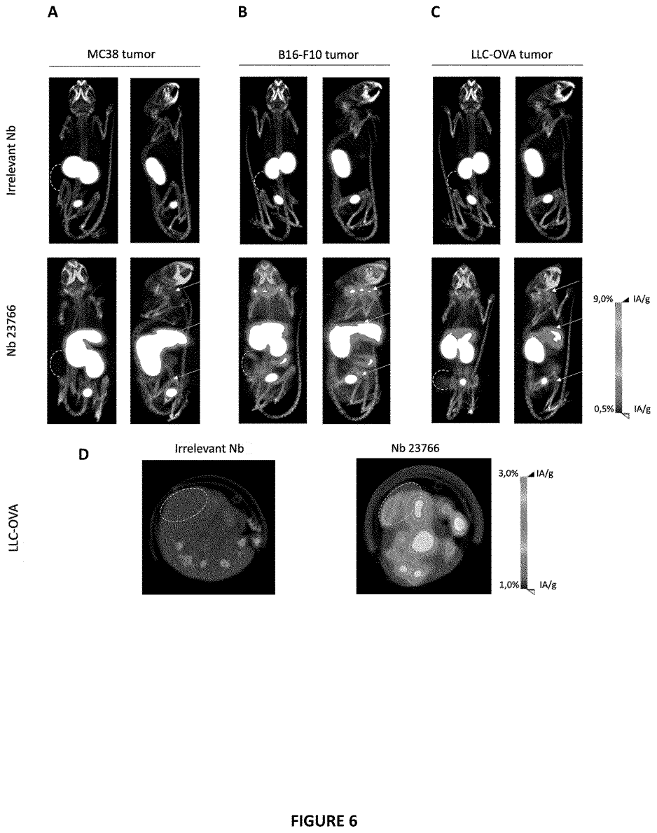

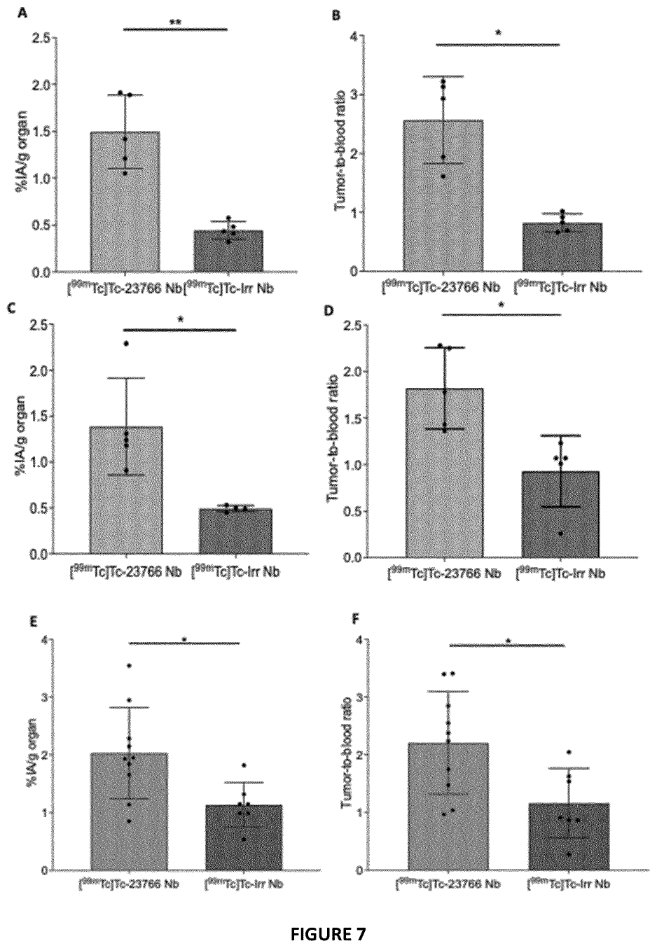

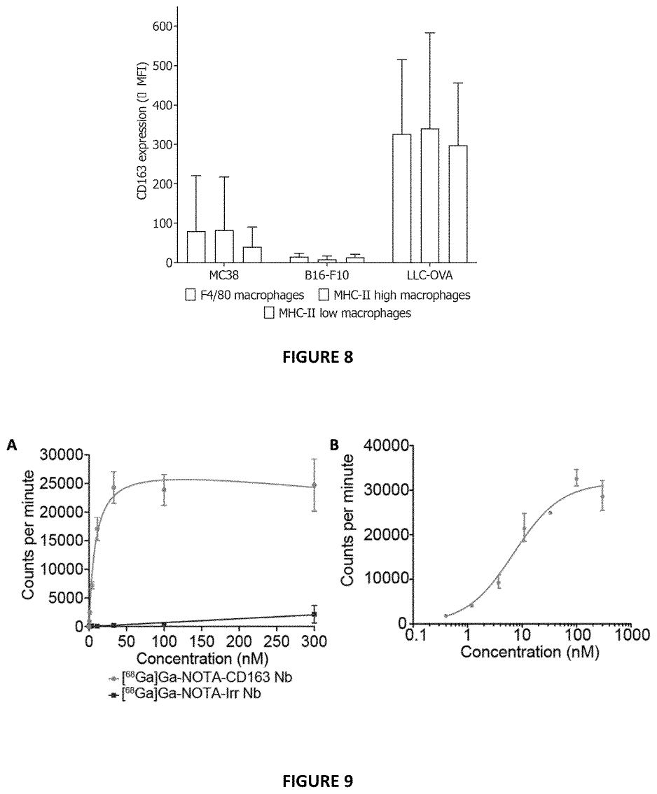

Next, we determined the macrophage specificity of the sdAb 23766 in untreated or macrophage-depleted mice. For comparison, we included sdAb 3.49, which targets the macrophage mannose receptor (MMR or CD206) and is currently in clinical trials (NCT04168528). One week prior to the biodistribution experiment, 3 mice received chow containing the colony-stimulating factor 1 receptor (CSF1R) inhibitor PLX3397, resulting in macrophage depletion, while the other 3 mice received control chow. Untreated mice showed high uptake in cervical lymph nodes, liver, and bone marrow for both anti-MMR and anti-CD163 sdAbs. Interestingly, while the sdAb 23766 signal in the liver significantly declined in mice treated with the PLX3397 compound, the anti-MMR sdAb signal in liver remained the same under these conditions. Hence, the anti-MMR sdAb does not only target macrophages but also other cells expressing the MMR receptor in the liver (e.g., LSEC), while the anti-CD163 sdAb is macrophage-specific (FIG. 5). We then performed biodistribution experiments in three tumor models (MC38, B16-F10 and LLC-OVA) as we aimed to visualize CD163-expressing tumor-associated macrophages (TAMs) with the sdAb 23766. Tumor cells were inoculated subcutaneously in the right flank of the animals 2-2.5 weeks prior to the imaging experiment to let the tumors grow until 500-1000 mm3. On the SPECT/CT images and the ex vivo biodistribution data, a limited uptake of the ^{99m}Tc-labeled sdAb 23766 was observed within MC38 tumors (FIG. 6A and FIG. 7A). The B16-F10 tumor model showed uptake of the radiolabeled sdAb in the periphery of the tumor (FIG. 6B) and similar ex vivo uptake as the MC38 tumor (FIG. 7C). In the case of the LLC-OVA tumor, ^{99m}Tc-labeled sdAb 23766 uptake was seen in the center of the tumor with a higher mean ex vivo uptake as compared to the other 2 tumor models (2% IA/g) (FIG. 7E). Moreover, the ^{99m}Tc-labeled sdAb 23766 showed significantly higher tumor-to-blood ratios in all three tumor models compared to the ^{99m}Tc-labeled irrelevant sdAb (FIG. 7B-D-F). The results obtained from the SPECT/CT images and ex vivo γ-counting could be validated by the flow cytometry data showing CD163 expression in the different tumors, with the LLC-OVA tumor demonstrating the highest CD163 expression values on TAMs recognized by the marker F4/80. The marker MHC-II was also included to determine the MHC-II low and MHC-II high TAM population in the immune cell compartment (FIG. 8).

### Example 3. The Lead Anti-CD163 Tracer is Converted Towards a PET Tracer

As we want to use the anti-CD163 immunotracer for nuclear imaging of patients, ideally the tracer needs to be converted to a PET tracer. This entailed the conjugation of the single domain antibody to a chelator as the radiometal ⁶⁸Ga will be used for radiolabeling. A final conjugation method resulted in a ratio of 2.32±0.12 chelator: single domain antibody as determined via mass spectrometry. Upon labeling, stability in injection buffer and human serum was evaluated at RT and 37° C. on different timepoints. After 60 min, the [⁶⁸Ga]Ga-NOTA-anti-CD163 single domain antibody was still stable in injection buffer (RCP; 95.5±1.2%) and human serum (RCP; 92.2±2.9%) (Table 3). The NOTA-single domain antibody displayed a similar binding affinity to recombinant hCD163 protein (K_{D}: 1.55±0.33 nM) and to HEK293T mCD163⁺ cells (K_{D}: 12.0±0.8 nM) indicating no effect of the NOTA-conjugation on binding properties. After radiolabeling, with either Gallium-68 (⁶⁸Ga) (t_{1/2}=68 min) or the longer-lived surrogate isotope Gallium-67 (⁶⁷Ga) (t_{1/2}=78.3 h), both the [⁶⁸Ga]Ga-NOTA-anti-CD163 single domain antibody and the [⁶⁷Ga]Ga-NOTA-anti-CD163 single domain antibody still showed a binding affinity in the low nanomolar range to hCD163 recombinant protein (K_{D}: 9.11±3.32 nM) (FIG. 9A) and to HEK293T mCD163⁺ cells (K_{D}: 7.82±1.13 nM) (FIG. 9B).

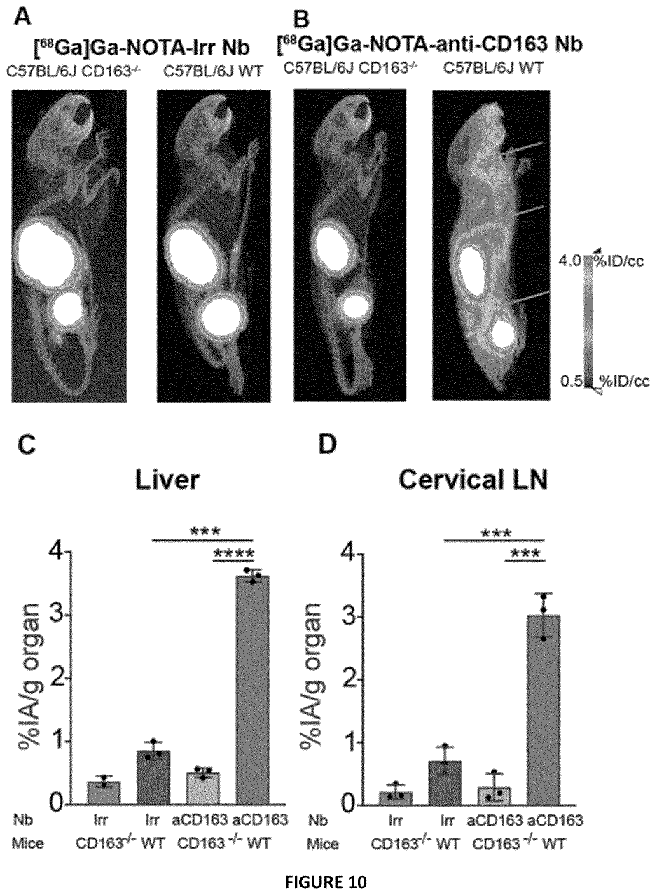

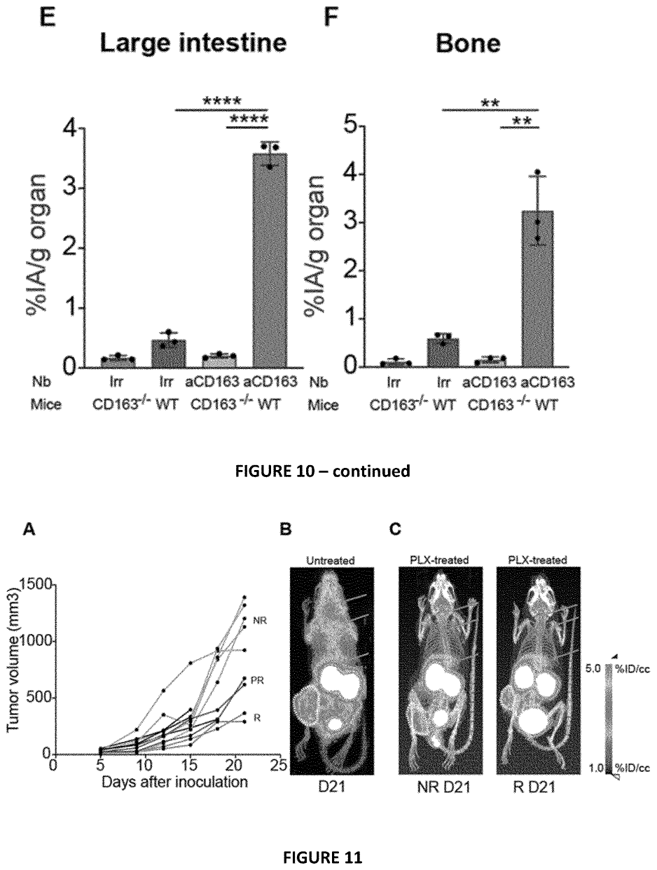

The PET tracer was again tested for CD163⁺ cell specificity via μPET/CT imaging and ex vivo analysis. The [⁶⁸Ga]Ga-NOTA-anti-CD163 single domain antibody and [⁶⁸Ga]Ga-NOTA-Irr single domain antibody (±10 MBq) were intravenously injected in naïve C57BL/6J WT and CD163^{-/-} mice (n=3). The anti-CD163 tracer shows specific radioactive uptake on the PET/CT images (FIG. 10A-B) and via ex vivo (v)-counting in cervical and inguinal lymph nodes, liver, intestines, and bone marrow (FIG. 10 C-F). Hence, we can conclude that the CD163 single domain antibody was successfully converted to a PET tracer.

### Example 4. The Lead Anti-CD163 Single Domain Antibody is Able to Visualize TAM Dynamics in the Tumor Microenvironment During CSF1R Therapy with Longitudinal Imaging

As the final goal is to longitudinally track TAMs inside the tumor microenvironment (TME) with the radiolabeled sdAb 23766 during immunotherapy, LLC-OVA tumor-bearing mice receive the macrophage-depleting compound PLX3397 in their food for 21 days (600 mg/kg AlN-76A chow). Control mice receive standard AlN-76A chow. During this period, mice are scanned on 3 different timepoints to determine the presence of CD163-expressing TAMs. A significantly lower uptake is seen on the SPECT/CT images and ex vivo γ-counting data in mice treated with PLX3397, in lymph nodes, liver and tumor. Flow cytometry data confirms a significant decrease of macrophages in tumor and liver in the PLX3397-treated group. The radiolabeled anti-CD163 sdAb 23766 is able to visualize distributions of TAMs during anti-macrophage immunotherapy.

### Example 5. Nuclear Imaging with the Lead Anti-CD163 Single Domain Antibody Tracer is Able to Noninvasively Monitor Macrophage Depletion in the Tumor Microenvironment During Anti-Macrophage Therapy

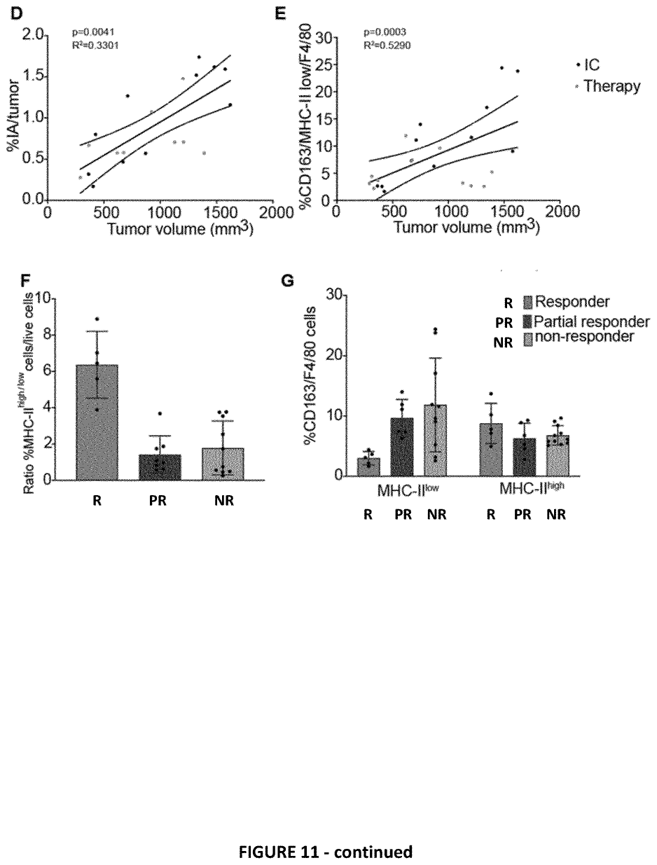

Finally, we set out to visualize therapy outcome via imaging of TAMs in the tumor microenvironment (TME) during immunotherapy using the radiolabeled anti-CD163 immunotracer. To this end, LLC-OVA tumor-bearing mice were either treated with a macrophage-depleting compound PLX3397 or control chow for 16 days. Interestingly, treated mice could be classified in different groups based on tumor growth in either non-responders (showing no effect on tumor growth), partial responders (showing partial tumor growth reduction) or responders (showing reduced tumor growth) (FIG. 11A). As expected, the anti-CD163 tracer showed high uptake in lymph nodes, bone marrow, liver, and tumor in untreated mice (FIG. 11 B). The uptake of the anti-CD163 tracer in lymph nodes, bone marrow, and liver of PLX-treated mice was lower as compared to untreated mice on PET/CT images (FIG. 11 B-C). The responder (R) mouse showed less overall uptake as compared to the non-responder mouse (FIG. 11 C). Significant correlations between tumor volume and radioactive uptake (% IA) as well as tumor volume and percentage of CD163⁺ MHC-II^{low} macrophages could be seen, which supported the PET imaging data (FIG. 11 D-E). Further, flow cytometry analysis indicated that tumors of responders contained a higher ratio of more MCH-II^{high}/MHC-II^{low} TAMs which is correlated with a more anti-tumoral phenotype of macrophages as compared to partial and non-responders (Wang et al. 2011, BMC immunology 12:43) (FIG. 11 F). The responders also showed less CD163 expression as compared to partial and non-responders (FIG. 11 G).

### Example 6. Materials and Methods

### 6.1. DNA Constructs

The lentiviral pHR vector, packaging vector pCMV packaging plasmid pCMVAR8.9 and the VSV.G encoding plasmid pMD.G were a gift from D. Trono (University of Geneva, Switzerland). PHR vectors encoding for the hCD163 or mCD163 protein were generated via in-Fusion cloning (Takara Bio, Kusatsu, Japan).

### 6.2. Cell Culture

HEK293T cells and B16-F10 cells were purchased from ATCC (Wesel, Germany). LLC-OVA cells were kindly provided by Dmitry Gabrilovich (The Wistar Institute, Philadelphia, USA). MC38 cells were kindly provided by Massimiliano Mazzone (VIB-KU Leuven, Belgium). All cells were grown at 5% CO₂ and 37° C. LLC-OVA cells were grown Roswell Park Memorial Institute (RPMI) 1640 Medium (Thermo Fisher Scientific, Waltham, Massachusetts, USA) supplemented with 1% Penicillin/Streptomycin (Gibco, Thermo Fisher Scientific) and 10% Fetal Bovine Serum (FBS, Serana, Pessin, Germany). HEK293T, MC38 and B16-F10 cells were grown Dulbecco's Modified Eagle's Medium (DMEM, Thermo Fisher Scientific) supplemented with 1% Penicillin/Streptomycin and 10% FBS.

### 6.3. Animal Models

Female wildtype C57BL6/J mice were purchased from Charles River (Ecully, France). CD163/mice have been described previously (Fischer-Riepe et al. 2020, J Allergy Clin Immunol 146:1137-1151) and were kindly provided by Johannes Roth (WWU Münster, Germany). In the case of imaging of tumor-bearing mice, mice were subcutaneously injected with MC38, B16-F10 or LLC-OVA tumor cells in the right flank.

Mice were examined daily, and tumor growth was measured using a digital caliper. Tumor volume was calculated using the formula (length×width2)/2. All experiments using mice were approved by the Ethical Committee for laboratory animals of the Vrije Universiteit Brussel and executed in accordance with the European guidelines for animal experimentation (ethical dossier numbers 21-272-14, 21-272-23 and 22-272-28).

6.4. sdAb Generation, Selection, and Production

Two llamas were subcutaneously injected 6 times with 100 μg recombinant human (h) CD163-Avi-His6 (U-Protein Express BV), 100 μg recombinant human CD163-His6 (Acro Biosystems, Newark, DE, USA), 100 μg recombinant mouse (m) CD163-Avi-His6 (U-Protein Express BV) and 100 μg recombinant mCD163-His6 (provided by Johannes Roth, WWU Münster), mixed with Gerbu adjuvant P (Gerbu Biotechnik) on a weekly basis. After immunizations, peripheral blood of both llamas was collected, and peripheral blood mononuclear cells were isolated using lymphoprep tubes (Greiner Bio-one, Kremsmunster, Austria). RNA was isolated from peripheral blood lymphocytes using RNA extraction kit (Qiagen, Hilden, Germany) and reverse transcribed into cDNA. Next, genes encoding for the variable domain of the heavy-chain only antibodies were amplified and ligated into the pMECS phage vector (Muyldermans 2021, FEBS J 288:2084-2102) resulting in 2 separate phage display libraries. Subsequent biopanning was performed by infection of the libraries with M13K07 helper phages, resulting in phage production. For each library, 3 rounds of panning in solution was performed using in-house site-specifically biotinylated hCD163-Avi-His6 or mCD163-Avi-His6. For round 1 and 2, 100 nM of antigen was used while 10 nM of antigen was used during the final round of panning. In total, 190 unique clones (95 from round 2 and 95 from round 3) were randomly selected and screened for their ability to specifically bind to hCD163 and mCD163 via ELISA. Specific binding was determined via ELISA using site-specifically biotinylated hCD163-Avi-His6 and mCD163-Avi-His6, immobilized on a streptavidin-coated 96-wells plate. Positive hits were sent for sequencing (Eurofins genomics) and grouped into different B cell lineages based on the CDR3 sequence. sdAbs were produced and purified as previously described (Pardon et al. 2014, Nat Protoc 9:674-693).

### 6.5. Surface Plasmon Resonance (SPR)

The affinity of purified CD163-targeting sdAb and of the NOTA-conjugated anti-CD163 sdAb to recombinant hCD163 and mCD163 protein (U-Protein Express BV) was determined using the BIACORE-T200 (GE Healthcare, Freiburg, Germany). Surface plasmon resonance measurements were performed at 25° C. with HEPES buffered saline (HBS, 20 mM of HEPES pH 7.4, 150 mM of NaCl, 3.4 mM of EDTA 0.05% Tween-20) running buffer. The sdAbs were injected consecutively in 2-fold serial dilutions, from 250 to 1 nM. The association step was 100 s, the dissociation step was 200 s, and the two-step regeneration of 35 s at 30 μL·min-1, using 100 mM glycine at pH 3.0, was performed. Local curve fitting analysis was performed using the BIACORE evaluation software (GE Healthcare) by fitting the obtained sensorgrams to theoretical curves, assuming 1-1 binding geometries. For the determination of the equilibrium dissociation constant, the ratio of the association and dissociation rate constants were determined.

### 6.6. Affinity Determination Via Flow Cytometry

Serial dilutions of the CD163-targeting sdAb and of the NOTA-conjugated anti-CD163 sdAb were incubated with 500.000 HEK293T cells overexpressing hCD163 or mCD163 in FACS buffer (HBSS (Gibco) supplemented with 1% FBS and 2 mM EDTA (Duchefa Biochemie, Haarlem, The Netherlands)) for 1 h at 4° C. NOTA-sdAb binding was detected by incubation of the cells with a PE tagged anti-VHH Ab (1:500 in FACS buffer, Genscript, Piscataway, NJ, USA) for 30 min at 4° C. Cells were washed once with FACS buffer. Next, sdAb binding was detected by incubation of the cells with an Alexa Fluor®-488 tagged anti-HA antibody (1:1000 in FACS buffer, clone 16B12, Biolegend, San Diego, CA, USA) for 30 min at 4° C. Again, cells were washed once with FACS buffer. sdAb binding was determined using the FACS CANTO II analyser (BD Biosciences, Franklin Lakes, NJ, USA). The mean fluorescent intensity of sdAb binding was determined using FlowJo version 10.

### 6.7. Thermal Shift Assay

sdAbs (concentration ranging 0.2 mg/ml to 0.5 mg/ml) were mixed with 1×SYPRO™ Orange Protein Gel Stain (Thermo Fisher Scientific) in PBS and added to white 96-well PCRs plates (Biorad, Pleasanton, CA, USA). Fluorescence signal was measured during increasing temperature steps ranging from 20 to 95° C., with stepwise increments of 0.5° C., using CFX connect™ Real-Time PCR (Biorad). Melting temperatures of the sdAbs was calculated using the Boltzmann equation.

### 6.8. Anti-CSF1R Therapy in Naïve and Tumor-Bearing Mice

To deplete macrophages in naïve and tumor-bearing mice, the CSF1R inhibitor Pexidartinib (PLX3397) is used. PLX3397 (AdvancedChemblock, Inc.) is incorporated in AlN-76A chow by Research Diets, Inc. at a concentration of 600 mg/kg chow. To achieve depletion, mice are given PLX3397 food ad libitum for 7 days in the case of naïve mice and 21 days in the case of LLC-OVA tumor-bearing mice. Control mice receive AlN-76A standard chow for the same period (n=3/group).

6.9. ^{99m}Tc-Radiolabeling of sdAbs

sdAbs were labeled with ^{99m}Tc as previously described (Xavier et al. 2012, Methods Mol Biol 911:485-490). Briefly, ^{99m}Tc-tricarbonyl was generated via the addition of 150 mCi ^{99m}Tc04 to the Isolink® labelling kit (Paul Scherrer Institute, Villigen, Switzerland) for 20 min at 100° C. Next, 50 μg of His-tagged sdAb was added and incubated for 90 min at 50° C. ^{99m}Tc-labeled sdAbs were purified via gel filtration from the unbound [^{99m}(H₂O)₃ (CO)₃]+ via a NAP-5 column (Cytiva, Machelen, Belgium) and filtered through a Millex 0.22 μm filter (Millipore, Haren, Belgium). The radiochemical purity of radiolabeled sdAbs was evaluated by instant layer chromatography (iTLC-SG, Pall Corporation, Hoegaarden, Belgium).

### 6.10. Pinhole SPECT-Micro-CT Imaging and Image Analysis

Mice were injected with approximately 5 μg of radiolabeled sdAb. One hour post injection, mice were anesthetized with 75 mg/kg ketamine and 1 mg/kg medetomidine (Ketamidor, Richter Pharma AG, Weis, Austria) via intraperitoneal injection and SPECT-Micro-CT Imaging was performed using a vector⁺ scanner (MiLABS, Houten, The Netherlands). Imaging set up consisted of a 1.5 mm 75-pinhole general purpose collimator, in spiral mode with 6 bed positions. Total SPECT scanning time was 15 min with 150 seconds per position and CT scanning (60 kV and 615 mA) was 2 min. After imaging, mice were killed, and organs were collected. Radioactivity in each organ was determined using a Wizard² γ-counter (Perkin-Elmer, Waltham, MA, USA). Uptake in each organ was corrected for radioactive decay and calculated as percentage of injected activity per gram of organ. SPECT/CT image analysis was performed using AMIDE (UCLA, CA, USA) and OsiriX (Pixmea, Geneva, Switzerland) software.

### 6.11. Processing Organs and Flow Cytometry Analysis

Single cell preparations of tumors and liver were prepared as described previously (Van Damme et al. 2021, J Immunother Cancer 9: e001749). Antibodies used for staining of single cell preparations can be found in Table 2. Delta median fluorescence intensity (AMFI) was determined via subtraction of the MFI of the staining and the MFI of the isotype control. Data was acquired using the FACS CANTO II analyser and analyzed using FlowJo.

6.12. Random Conjugation of the NOTA Chelator to the Lysines of the sdAb and Lon Exchange

The random conjugation of the sdAb to p-SCN-Bn-NOTA (NOTA-NCS, Macrocyclics, Inc., Plano, TX, USA) was adjusted in comparison to the standard protocol (Xavier et al. 2013, J Nucl Med 54:776-784) in order to receive the most optimal chelator-to-sdAb ratio. The anti-CD163 sdAb (7 mg, 0.49 μmol) at a concentration of 5.5 mg/mL was first buffer-exchanged to 0.25 M sodium carbonate adjusted to pH 9.75 (sodium carbonate anhydrous; sodium hydrogen carbonate; sodium chloride, VWR Chemicals, Leuven, Belgium) using a PD-10 size exclusion column (GE Healthcare, Buckinghamshire, UK). A 30-fold molar excess of NOTA-NCS was added to the sdAb solution (7.98 mg, 1.43 μmol) and incubated for 3 h 30 at RT. After incubation, the NOTA-sdAb was purified via size exclusion chromatography (SEC) on a Hiload™ 16/600 Superdex™ 30 μg column (GE Healthcare Bio-Sciences AB, Uppsala, Sweden) with 0.1 M NaOAc as a mobile phase (0.8 mL/min) to separate the conjugated sdAb from excess NOTA-NCS. The concentrations of the collected NOTA-sdAb fractions were measured spectrophotometrically using Nanodrop 2000 by UV absorption at 280 nm (E=72287 M−1cm−1, MW=15309 Da). SEC with a Superdex Peptide 10/300 GL column (GE Healthcare Bio-Sciences AB, Uppsala, Sweden) was also used for quality control of the NOTA-sdAb. The number of chelators per sdAb was determined by electrospray ionization quadrupole time-of-flight mass spectrometry (ESI-Q-TOF-MS).

6.13. ^{68/67}Ga-Labeling of the NOTA-CD163 sdAb and Radiometal Chelation Stability Assessment

The randomly conjugated NOTA-CD163 sdAb (6.5 nmol) was added to 1 ml of 1 M NaOAc buffer pH 5 and 1 mL of ⁶⁸Ga eluate (374-618 MBq) eluted from a ⁶⁸Ge/⁶⁸Ga generator in 0.1 M HCl (Galli Eo™, IRE ELIT, Fleurus, Belgium) and incubated for 10 min at RT. Purification was performed on a PD-10 desalting column pre-equilibrated with 1×PBS in case of test-labeling or 0.9% NaCl containing 5 mg/ml vitamin C pH 5.8-6.1 (injection buffer) in case of stability and in vivo studies. After purification, the radioactive sdAb solution was filtered through a 0.22 μm filter (Millipore, Belgium). The radiochemical purity was assessed before and after purification by radio-iTLC ([⁶⁸Ga]Ga-NOTA-sdAb Rf=0, [⁶⁸Ga]Ga-citrate Rf=1). The cell binding study required long incubation and washing steps, so the NOTA-sdAb was labeled with the longer-lived isotope ⁶⁷Ga instead of ⁶⁸Ga [⁶⁷Ga]GaCl₃ was obtained from [⁶⁷Ga]Ga-citrate by diluting the solution with metal free water (TraceSELECT™, Honeywell Riedel-de Haen™ Fisher Scientific) and adding the final solution to a Waters Sep-Pak® Reservoir adaptor. The sdAb labeling required 6.5 nmol of NOTA-sdAb, 5M NH4OAc pH 5-5.2, and #111 MBq of [⁶⁷Ga]GaCl3. A NAP-5 column (GE Healthcare, Belgium) was used to purify the radiolabeled sdAb solution and was followed by filtration.

Affinity determination of the [⁶⁸Ga]Ga-NOTA-anti-CD163 sdAb via ELISA was performed as described in the previous paragraph. The radioactive sdAb binding on the hCD163-Avi-His6 protein was measured via a γ-counter.

Radiometal chelation stability of the [⁶⁸Ga]Ga-NOTA-anti-CD163 sdAb (5-69 MBq, after filtration) was assessed in different conditions (injection buffer RT, 37° C.; human serum 37° C.; mouse serum 37° C.) at 30 min, 60 min, 120 min and 180 min after labeling. Stability of the radiolabeled compound was analyzed via radio-iTLC and radio-SEC at these timepoints.

6.14. Cell Binding Study with [⁶⁷Ga]Ga-NOTA-Anti-CD163 sdAb 5×10⁴ HEK293T mCD163⁺ cells in 1 mL of DMEM medium per well were plated out in a 24-well plate 2 days prior to the radioactive cell binding study. The plate was cooled to 4° C. one hour prior to experiment.

After 30 min, the plates were taken out and medium was replaced with 400 μL unsupplemented medium or blocking unsupplemented medium (100 molar excess of unmodified sdAb). Radiolabeled sdAbs in different concentrations ranging from 300 nM to 0.1 nM were added to the cells. After a 1 h incubation at 4° C., wells were washed twice with 0.75 mL ice-cold 1×PBS and lysis was performed by adding 0.75 ml 1M NaOH at RT for 5 min. All fractions were collected and counted in the Wizard² γ-counter (PerkinElmer, Mechelen, Belgium).

### 6.15. PET/CT Imaging and Image Analysis

Timing of injections and use of anesthesia are identical to section ‘Pinhole μSPECT/CT Imaging and Image Analysis’. C57BL/6J WT and C57BL/6J CD163/mice were injected with [⁶⁸Ga]Ga-NOTA-sdAb (92.7±17.5 MBq/mL, 0.33 nmol) in a volume of 130-170 μL. Mice were imaged in prone position and fixated in a mouse hotel or single mouse bed with iFIX Fleece 5 tape (Interventional systems, Kitzbühel, Austria) and imaged for 12-20 min using a Molecubes PET-cube/CT X-cube system (Molecubes, Ghent, Belgium) with a resolution of 850 μm. The Molecubes PET system includes 45 PET detectors arranged in 5 rings to provide a scanner diameter of 7.6 cm and axial length of 13 cm (Krishnamoorthy et al. 2018, Phys Med Biol 63:155013). The energy peak was set on 511 keV and the energy resolution on 30%. Reconstruction was performed on the software of Molecubes using an OSEM algorithm and attenuation was based on the CT image. The tumor of the mouse at the right flank was positioned at 7-7.5 cm in the field of view (FOV). A phantom-based calibration of the scanner was performed with a 500 ML syringe with a minimum of 20 μCi at the axial center of the FOV. Postprocessing was performed with VivoQuant™ 2022 (Invicro, Needham, USA). The resulting radioactive concentration was measured per tissue volume (Becquerel/cubic centimeter) decay-corrected and presented as percentage of injected dose per cubic centimeter (% ID/cc).

### 6.16. Statistical Analyses

Quantitative data are presented as mean±SD and were analyzed using GraphPad Prism (version 9.5.1) software. Statistical analyses were performed using an unpaired two-tailed t-test, one-way ANOVA with Dunnett's multiple comparisons test or a two-way ANOVA with Dunnett's multiple comparisons test and this is specified in the figure legends. P<0.05 was considered as statistically significant, with ns, p>0.05; *, p<0.05; **, p<0.01; ***, p<0.001; ****, p<0.0001.

TABLE 2
Overview antibodies used for flow cytometry

| Target | Fluorophore | Species reactivity | Provider | Clone |
|---|---|---|---|---|
| CD11b | PE/Cyanine7 | Human, Mouse | Biolegend | M1/70 |
| CD45 | APC/Cyanine7 | Mouse | Biolegend | 30-F11 |
| SiglecF | PE | Mouse | Biolegend | S17007L |
| Ly6G | PE | Mouse | Biolegend | 1A8 |
| Ly6C | PerCP/Cyanine5.5 | Mouse | eBioscience | HK1.4 |
| F4/80 | FITC | Mouse | Biolegend | BM8 |
| MHC-II | BV421 | Mouse | Biolegend | M5/114.15.2 |
| CD206 | APC | Mouse | Biolegend | C068C2 |
| CD206 isotype | APC | / | Biolegend | RTK2758 |
| control | | | | |
| CD163 | APC | Mouse | Biolegend | TNKUPJ |
| CD163 isotype | APC | / | Biolegend | RTK2758 |
| control | | | | |
| VHH | PE | Camelid | Genscript | / |
| CD69 | PE/Cyanine7 | Mouse | Biolegend | H1.2F3 |
| CD8 | Brilliant violet 421 | Mouse | Biolegend | 53-6.7 |

TABLE 3
Radiochemical purities of the ⁶⁸Gallium-labeled anti-CD163
single domain antibody in a stability study. The radiochemical
purity (RCP) of the ⁶⁸Gallium-labeled anti-CD163 single domain
antibody at different timepoints and conditions. Data presented
as mean ± S.D. of at least 3 independent experiments.

| Timepoint | RCP Human serum (%) | RCP Injection buffer (%) |
|---|---|---|
| T0, 37° C. | N.D. | 98.7 ± 1.3 |
| T30, 37° C. | 94.8 ± 1.3 | 96.6 ± 1.8 |
| T60, 37° C. | 94.8 ± 1.6 | 95.5 ± 1.2 |
| T120, 37° C. | 88.4 ± 2.8 | 94.4 ± 1.7 |
| T180, 37° C. | 84.1 ± 2.8 | 92.0 ± 2.8 |
| T180, RT | N.D. | 96.6 ± 2.0 |

1. A single domain antibody (sdAb) comprising a CDR1 region, a CDR2 region, and a CDR3 region, wherein the CDR1, CDR2 and CDR3 regions are selected from those CDR1, CDR2 and CDR3 regions as present in an immunoglobulin variable domain (IVD) defined by SEQ ID NO:1 and as determined by the Kabat, Chothia, Martin, or IMTG method.

2-15. (canceled)

16. The single domain antibody (sdAb) according to claim 1 wherein the CDR1 region is defined by SEQ ID NO: 2, the CDR2 region is defined by SEQ ID NO: 3, and the CDR3 region is defined by SEQ ID NO:4.

17. The single domain antibody (sdAb) according to claim 1 wherein the CDR1 region is defined by SEQ ID NO: 2, the CDR2 region is defined by SEQ ID NO: 3, and the CDR3 region is defined by SEQ ID NO: 4, as determined by the Kabat method.

18. The single domain antibody (sdAb) according to claim 1 further comprising at least an FR1, FR2, FR3, or FR4 region as present in the IVD defined by SEQ ID NO: 1.

19. The single domain antibody (sdAb) according to claim 1, further comprising FR1, FR2, FR3, or FR4 region as present in the IVD defined by SEQ ID NO: 1; wherein the FR1 region is defined by SEQ ID NO: 5, the FR2 region is defined by SEQ ID NO: 6, the FR3 region is defined by SEQ ID NO: 7, and the FR4 region is defined by SEQ ID NO: 8.

20. The single domain antibody (sdAb) according to claim 1, comprising SEQ ID NO: 1.

21. The single domain antibody (sdAb) according to claim 1, wherein the CDR and/or FR regions are humanized and/or the IVD is humanized.

22. The single domain antibody (sdAb) according to claim 1, further comprising a functional moiety.

23. The single domain antibody (sdAb) according to claim 1, further comprising a functional moiety, selected from the group consisting of a His-tag, a peptide motif recognized by a peptide ligase, and a detectable moiety.

24. The single domain antibody (sdAb) according to claim 1, which is conjugated to a prophylactic or therapeutic drug, to a cytotoxic moiety or drug, to an immunostimulatory or immunosuppressive agent, to a Toll-like receptor agonist, to a photon absorber, to a liposome or to a nanoparticle.

25. The single domain antibody (sdAb) according to claim 1 being in a pharmaceutical composition.

26. A method of treating or inhibiting progression of cancer in a subject, the method comprising administering to a subject the single domain antibody (sdAb) according to claim 1.

27. The method according to claim 26, wherein the single domain antibody (sdAb) is conjugated to a prophylactic drug, a therapeutic drug, or a cytotoxic moiety.

28. A method of using the single domain antibody (sdAb) according to claim 1 as an imaging agent, the method comprising administering to a subject the single domain antibody (sdAb) according to claim 1, followed by detection of CD163 expressing cells.

29. The method according to claim 28, wherein the imaging is for diagnosis or therapy monitoring.

30. The method according to claim 28, wherein the single domain antibody (sdAb) is coupled to a detectable moiety.

31. The method according to claim 28, wherein the single domain antibody (sdAb) is coupled to a detectable moiety selected from the group consisting of radioemitters/radiolabels selected from ⁶⁸Ga, ¹¹⁰In, ¹⁸F, ⁴⁵Ti, ⁴⁴Sc, ⁴⁷Sc, ⁶¹Cu, ⁶⁰Cu, ⁶²Cu, ⁶⁶Ga, ⁶⁴Cu, ⁵⁵Ca, ⁷²As, ⁸⁶Y, ⁹⁰Y, ⁸⁹Zr, ¹²⁵I, ⁷⁴Br, ⁷⁵Br, ⁷⁶Br, ⁷⁷Br, ⁷⁸Br, ¹¹¹In, ^{114m}In, ¹¹⁴In, ^{99m}Tc, ¹¹C, ³²Cl, ³³Cl, ³⁴Cl, ¹²³I, ¹²⁴I, ¹³¹I, ¹⁸⁶Re, ¹⁸⁸Re, ¹⁷⁷Lu, ⁹⁹Tc, ²¹²Bi, ²¹³Bi, ²¹²Pb, ²²⁵Ac, ¹⁵³Sm, and ⁶⁷Ga, fluorescence emitters selected from cyanine dyes, indolenine-based dyes, benzoindolenine-based dyes, phenoxazines, BODIPY dyes, rhodamines, Si-rhodamines, Alexa dyes, and derivatives of any thereof.

32. The method according to claim 28, wherein detection of CD163 expressing cells is by a method selected from the group consisting of PET (positron emission tomography), SPECT (single-photon emission computed tomography), fluorescence imaging, fluorescence tomography, near infrared imaging, near infrared tomography, and optical tomography.

33. The method according to claim 28, wherein the method is for cancer or tumour imaging.

34. The method according to claim 28, wherein the method is for monitoring of macrophage depletion in a tumour microenvironment.


SEQUENCE LISTING (10 sequences)

SEQ ID NO: 1 - protein, 127 aa, synthetic construct
DVQLVESGGGLVQPGGSLRLSCAASGITFSSYAVAWFRQASGKEREFVAFIGWDGDTTYYVDSVKGRFTISRDNAKNMVYLQMNSLKPDDTAIYYCARHKTLWRSSWDNRPVQYDYWGQGTQVTVSS

SEQ ID NO: 2 - protein, 8 aa, synthetic construct
GITFSSYA

SEQ ID NO: 3 - protein, 8 aa, synthetic construct
IGWDGDTT

SEQ ID NO: 4 - protein, 20 aa, synthetic construct
ARHKTLWRSSWDNRPVQYDY

SEQ ID NO: 5 - protein, 25 aa, synthetic construct
DVQLVESGGGLVQPGGSLRLSCAAS

SEQ ID NO: 6 - protein, 17 aa, synthetic construct
VAWFRQASGKEREFVAF

SEQ ID NO: 7 - protein, 38 aa, synthetic construct
YYVDSVKGRFTISRDNAKNMVYLQMNSLKPDDTAIYYC

SEQ ID NO: 8 - protein, 11 aa, synthetic construct
WGQGTQVTVSS

SEQ ID NO: 9 - protein, 5 aa, synthetic construct
LPXTG

SEQ ID NO: 10 - protein, 5 aa, synthetic construct
LPETG